(19) United States
(12) Patent Application Publication
(10) Pub. No.: US 2026/0137562 A1
(43) Pub. Date: May 21, 2026

(54) MEDICAL BANDAGE BACKING SHEET

(71) Applicant: Dimitrije Stojanovki, Shelby Township, MI (US)

(72) Inventor: Dimitrije Stojanovki, Shelby Township, MI (US)

(21) Appl. No.: 18/952,763

(22) Filed: Nov. 19, 2024

(51) Int. Cl. A61F 13/0246

(52) U.S. Cl. CPC A61F 13/0253; A61F 13/025

(57) ABSTRACT

Standard backing sheets on bandages are made to secure the placement of the absorption pad and to prevent blood from seeping out. With these standard bandages the blood will accumulate in a single area from the wound site. This buildup in a single area will cause more frequent bandage replacements and it can cause seepage issues. The buildup can also cause a more damp and wet feeling for the user.

The improved medical bandage backing sheet can prevent this and distribute the blood from a wound site throughout that absorption pad. Utilizing the maximum surface area of that absorption pad or gauze and prevent blood build up in one certain area. In return causing less seepage, a damp feeling to the user, and bandage replacements. The improved backing sheet can also be made with oligodynamic coated threads giving it antimicrobial properties.

### TECHNICAL FIELD

The present disclosure relates generally to bandages for protection of injured skin. Examples of the present disclosure are related to bandages manufactured with antimicrobial, antifungal, and antibacterial metals for wound treatment.

### BACKGROUND

The statements in this section merely provide background information related to the present disclosure. Accordingly, such statements are not intended to constitute an admission of prior art.

A bandage is a strip of material used to protect, immobilize, compress, or support a wound or injured body part. Bandages are available in a wide range of types, from cloth strips, rolls, to specialized shaped bandages designed for specific body parts or types of injuries. Dressings or gauze are materials that are applied directly to wounds to promote healing and prevent further harm to the site of injury.

Typical bandages found in first-aid kits are strips made of plastic, fabric, or other suitable materials, with an adhesive side which is placed on the skin. An absorbent pad adhered on the adhesive side which is placed directly over the injured skin. Typical absorbent pads are made from cotton, polyester or other suitable materials. Other bandages consist of strips of material alone, which do not adhere to the skin but cohere to themselves, for use with separate absorbent pads.

Touchscreen devices employ electronic visual displays that the user can control by touching the screen with a finger or other object such as a stylus. Touchscreens are common in devices such as tablet computers and smartphones. Many touchscreens employ technology that requires an electrically conductive object to touch the screen for the user to be able to use the touchscreen device. Human skin is electrically conductive and can be used to interact with touchscreen devices.

However, certain circumstances arise in which skin must be kept covered. For example, when skin is injured, it is recommended that the skin be kept covered with a bandage. In such circumstances, the wound covering prevents electrically conductive skin from coming into direct contact with touchscreen devices that employ conductive technology, and therefore touchscreen devices can be used only with difficulty when skin must remain covered.

As they are currently manufactured, typical bandages cannot be used with touchscreen devices, as they lack the electrically conductive properties to do so. Studies have been performed on silver dressings. Some studies have shown improvements in wound healing times and healthful results when silver is in contact with a wound site during the healing process.

The oligodynamic effect is a biocidal effect of metals, especially heavy metals, which occurs even in low concentrations. This effect is attributed to the antibacterial behavior of metal ions, which are absorbed by bacteria upon contact and damage their cell membranes. Zinc, tin, brass, silver, gold, nickel, copper, and platinum are some of the most common oligodynamic metals known with antimicrobial, antifungal, and antibacterial properties. These metals also have conductive properties.

U.S. Patent Number US20230069052-A1 titled “metallic bandage” for which the present applicant was the sole inventor discloses previous known method of bandages with oligodynamic concentration. A bandage includes a backing sheet portion including a first oligodynamic treatment including a first relatively higher oligodynamic concentration. The bandage further includes an absorbent pad portion provided on the bottom surface of the backing sheet portion. The absorbent pad portion includes a second oligodynamic treatment including a second relatively lower oligodynamic concentration. The second relatively lower oligodynamic concentration is lower than the first relatively higher oligodynamic concentration. The second oligodynamic treatment is operable to release oligodynamic ions directly to the wound site. The first oligodynamic treatment enables the backing sheet portion to provide a reservoir of oligodynamic particles that may propagate outwardly from the bandage overview.

U.S. Patent Number US20230069052 mentions oligodynamic by concentration but not by coating thickness and coated thread width. For example, silver coated fabrics are a prior art that has been known in various industries. These fabrics have a very thick coating due to the frequent use of the fabric on medical or even electrical components. The thick coating makes these fabrics very stiff, minimum flexibility, and usually have a course/rough texture feel on the fabric. Some of these fabrics are even made with thin solid silver wires.

Some medical fabrics, devices, clothing, and cleaning devices also include silver coating on medical fabric and utensils. Many of these products are meant for heavy use of the device, long durations, and a longer shelf-life. Prior art on silver medical fabric, medical tape and absorption pads are either made with a low silver amount that have low to no conductivity. Other prior art has a heavy silver amount directly applied to the wound.

Adhesive bandages and gauze pads classified as a class 1 or 2 device with the FDA are usually changed daily. With applications under 48 hours there is no need to use a thick oligodynamic coating on the bandage. Applications with 6 microns or less for short-term, 6-20 for moderate use, and 20 microns or more for severe wear resistance applications.

For disposable applications under 24 or 48 hours greater than 0.02μ (microns) and less than 6μ (microns) will help keep manufacturing costs lower. The application will still have an essential amount of antimicrobial and conductive properties to it. Furthermore, the application will still maintain some flexibility to it. The thicker the coating the higher the manufacturing cost and less flexible the fabric is. Many bandages classified with the FDA as a class 1 or class 2 device are meant to be used for under 24 or 48 hours. Having a product with a thicker oligodynamic coating for frequent use is not necessary for disposable products under 24 or 48 hours. After several prototypes of various coating processes, electro-plating silver threads have better performance over other various coating processes. Less flaking and peeling of the threads happen with the electro-plating process. Better conductivity, antimicrobial properties and longevity of the threads are also associated with electro-plating.

Some prior art uses silver nano wire, flakes or spheres as the coating process. These nano-coating are 0.02 microns or less in thickness. Even though this coating falls under the short-term life span of the product. Coatings with 0.02 microns or less in thickness do not provide enough conductive properties needed to have the bandage be smart device compatible. Furthermore, 0.02 microns or less coating thickness is not as effective and efficient in killing germs then other coating thicknesses. In addition, 0.02 microns or less coating can cause the nano particles to flake or peel off easier when the bandage is being used. A simple procedure like washing your hands can affect the nano particle coating.

Some silver nano coatings have an additional Polyethylene terephthalate (PET) coating added over the silver nano-particle coating. This coating prevents flaking and peeling but also has some downfalls. Polyethylene terephthalate layer can increase manufacturing costs, lower the antimicrobial efficiency of the silver nano-coating, and release micro plastics into the body. Using a too thin of a coating, for example 0.02 microns or less can cut manufacturing costs, but you lose several advantages over a heavier coating. The antimicrobial, antifungal, and antibacterial of oligodynamic threads of 0.02 microns or less has a slower germ-killing rate that would take a lot longer to kill germs over a 24-48-hour period. If 0.02 microns or less coating was placed on a backing sheet there would be an exceptionally low conductivity rate or no conductivity at all on the bandage. Having conductive properties is beneficial for healing, heat transfer, and giving the user the ability to use the bandage on touchscreen devices.

Various coating processes are available for making silver or other oligodynamic coated materials. Some of these processes are electroless plating layer, a sputtering deposit layer, a dip coating layer, silver ink, and a nano-coating layer. The applications backing sheet fabric can be made from a single coated thread next to a non-coated thread. Prototype tests showed coated silver threads group together with a diameter width from 0.05 mm-0.6 mm then to have more flexibility and less of a stiff course feeling to it. Various testing has shown this range is ideal for a backing sheet for daily use for disposable bandages on class 1 and class 2 FDA devices. For applications that need a heavier long-term use with a stiffer backing sheet, silver threads group together with a width over 0.6 to 2 mm can be used.

### BRIEF DESCRIPTION OF THE DRAWINGS

One or more embodiments will now be described, by way of example, with reference to the accompanying drawings, in which:

FIGS. 1A-1C Illustrates prior art of a product called SilverSeal hydrogel burn & wound dressing with silver in it.

FIGS. 2A-2C Illustrates prior art of a product called Silver Calcium Alginate Dressing by Ceeport.

FIGS. 3A-3C Illustrates prior art of a product called Silver Alginate Dressing by Dimora

FIG. 4 Illustrates prior art of an absorption pad magnified in two separate views showing silver flakes on an absorption pad.

FIGS. 5A-5B & 5C Illustrates prior art of an adhesive bandage by Daker with a silver film on an absorption pad.

FIGS. 6-8 Illustrate prior art silver nanowires coatings

FIG. 9 Illustrates prior art various silver nanoparticle types

FIGS. 10A, 10B & 10C illustrate prior art, FIG. 10A illustrates a side view, 10B illustrates the top view. FIG. 10C illustrates the bandage of FIG. 10A including ends of the metallic fiber creating poke obstacles for a user, in accordance with the present disclosure.

FIGS. 11A & 10B illustrates an image of an oligodynamic coated on a thread in accordance with the present disclosure

FIG. 12 illustrates front view of a fabric adhesive bandage with an oligodynamic coated backing sheet in accordance with the present disclosure

FIG. 13 illustrates back view of a fabric adhesive bandage with an oligodynamic coated backing sheet and with an island type absorption pad in accordance with the present disclosure

FIG. 14 illustrates back view of a fabric adhesive bandage with an oligodynamic coated backing sheet with a fold edge showing flexibility of the bandage in accordance with the present disclosure

FIG. 15 illustrates front view of a fabric adhesive bandage with an oligodynamic coated backing sheet in accordance with the present disclosure

FIG. 16 illustrates back view of a fabric adhesive bandage with an oligodynamic coated backing sheet and with a non-island type absorption pad in accordance with the present disclosure

FIG. 17 illustrates side plan view of a fabric adhesive bandage with an oligodynamic coated backing sheet in accordance with the present disclosure

FIG. 18 illustrates end plan view of a fabric adhesive bandage with an oligodynamic coated backing sheet in accordance with the present disclosure

FIGS. 19-20 illustrates a front view of a fabric adhesive bandage with an oligodynamic coated backing sheet with an image magnified view of the coated threads on the backing sheet in accordance with the present disclosure.

FIG. 21 illustrates an image front view and a magnified view of the threads of prior art of a fabric adhesive bandage without any oligodynamic coating on the backing sheet

FIG. 22 illustrates images of two front views of fabric adhesive bandages next to one another, where label A image illustrates prior art of a fabric adhesive bandage without any oligodynamic coating on the backing sheet. Label B illustrates fabric adhesive bandage with and an oligodynamic coating on the backing sheet in accordance with the present disclosure.

FIGS. 23-30 illustrates front view of a fabric adhesive bandage with an oligodynamic coated backing sheet with an image magnified view of the coated threads on the backing sheet in accordance with the present disclosure.

FIG. 31. illustrates an image of a magnified view of the oligodynamic coating of the threads on the backing sheet of a fabric adhesive bandage in accordance with the present disclosure.

FIG. 32. illustrates an image of a magnified view of the oligodynamic coating of the threads on the backing sheet of a fabric adhesive bandage with a ruler showing threads width in accordance with the present disclosure.

FIG. 33A illustrates a magnified view of an oligodynamic coated thread in accordance with the present disclosure.

FIG. 33B illustrates a bundle of strands or fibers infused collectively with oligodynamic material, in accordance with the present disclosure.

FIG. 33C illustrates a bundle of strands or fibers coated on a periphery with oligodynamic material, in accordance with the present disclosure.

FIG. 33D illustrates a bundle of strands or fibers with some of the strands around a periphery of the bundle coated with oligodynamic material, in accordance with the present disclosure.

FIG. 33E illustrates a bundle of strands or fibers with some of the strands around a periphery of the bundle and in a center of the bundle are coated with oligodynamic material, in accordance with the present disclosure.

FIG. 33F illustrates a bundle of strands or fibers with strands not coated with oligodynamic material surrounding a core of a fiber or multiple fibers coated with oligodynamic material, in accordance with the present disclosure.

FIG. 34A Illustrates an oligodynamic backing sheet with the top layer has a coating on it, in accordance with the present disclosure.

FIG. 34B Illustrates an oligodynamic backing sheet with the top and bottom layer has a coating on it, in accordance with the present disclosure.

FIG. 35A-35E Illustrates various threads spun together with an oligodynamic coating on it, in accordance with the present disclosure.

FIG. 36. Illustrates images of two fabric adhesive bandages next to one another in petri dishes, where label A image illustrates prior art of a fabric adhesive bandage without any oligodynamic coating on the backing sheet. Label B illustrates fabric adhesive bandage with an oligodynamic coating on the backing sheet in accordance with the present disclosure.

FIG. 37. Shows prior art of various adhesive bandage shapes, forms that a fabric adhesive bandage with and an oligodynamic coating on the backing sheet can take shape in accordance with the present disclosure.

FIG. 38A. Shows and image of prior art of a medical gauze pad

FIG. 38B. Shows and image of prior art of a medical gauze pad in a roll FIG. 38C. Shows a sketch of prior art of a medical gauze pad

FIG. 39. illustrates front view of a medical gauze pad with an oligodynamic coated backing sheet in accordance with the present disclosure.

FIG. 40. illustrates back view of a medical gauze pad with an oligodynamic coated backing sheet in accordance with the present disclosure.

FIG. 41A. illustrates side view of a medical gauze pad with an oligodynamic coated backing sheet with the present disclosure.

FIG. 41B. illustrates side view of a medical gauze pad with an oligodynamic coated backing sheet with an extended periphery with the present disclosure

FIG. 41C. illustrates side view of a medical gauze pad with an oligodynamic coated backing sheet with an extended periphery and with a gauze pad located at the top and bottom in accordance with the present disclosure.

FIG. 41D. illustrates side view of a medical gauze pad with an oligodynamic coated backing sheet and gauze pad located at the top and bottom in accordance with the present disclosure.

FIG. 41E. illustrates side view of a medical gauze pad with an oligodynamic coated backing sheet with a pattern design absorption pad attached at the bottom in accordance with the present disclosure.

FIG. 41F. illustrates side view of a medical gauze pad with an oligodynamic coated backing sheet with a stacked of absorption pad and backing sheet with the present disclosure.

FIG. 41G. illustrates side view of a medical gauze pad with an oligodynamic coated backing sheet with a pattern design absorption pad attached at the bottom and top. Also illustrates a pattern and an offset pattern of the gauze pad in accordance with the present disclosure.

FIG. 42. Illustrates a medical gauze pad with an oligodynamic coated backing sheet with a pattern diamond design absorption pad attached in accordance with the present disclosure.

FIG. 43. Illustrates a square medical gauze pad with an oligodynamic coated backing sheet with a pattern hexagon design absorption pad attached in accordance with the present disclosure.

FIG. 44. Illustrates a medical gauze pad with an oligodynamic coated backing sheet with a pattern checkered design absorption pad attached in accordance with the present disclosure.

FIG. 45. Illustrates a medical gauze pad with an oligodynamic coated backing sheet with a double circular pattern absorption pad design attached in accordance with the present disclosure.

FIG. 46. Illustrates a medical gauze pad with an oligodynamic coated backing sheet with a singular circular pattern absorption pad design attached in accordance with the present disclosure.

FIG. 47. Illustrates a medical gauze pad with an oligodynamic coated backing sheet with a multiple circular pattern absorption pad design attached in accordance with the present disclosure.

FIGS. 48, 49A & 49B Illustrates oligodynamic backing sheet with perforation on the backing sheet and the various perforation shapes in accordance with the present disclosure.

FIG. 50 Illustrates distribution of fluids due to the oligodynamic backing sheet perforation, in accordance with the present disclosure.

FIGS. 51-54 Illustrates oligodynamic backing sheet with segmented structure, the various perforation shapes, spacings and distribution of fluids in accordance with the present disclosure.

FIG. 55 Illustrates oligodynamic backing sheet made in segmented lines, horizonal lines, vertical lines, diagonal lines, curves, crosses, zig zag, circles, and waves with the present disclosure.

FIG. 56 Illustrates an oligodynamic backing sheet made in various shapes, characters, illustrations etc. in accordance with the present disclosure.

Corresponding reference characters indicate corresponding components throughout the several views of the drawings. Skilled artisans will appreciate that elements in the figures are illustrated for simplicity and clarity and have not necessarily been drawn to scale. For example, the dimensions on some of the elements in the figures may be exaggerated relative to other elements to help to improve understanding of various embodiments of the present disclosure. Also, common but well-understood elements that are useful or necessary in a commercially feasible embodiment are often not illustrated to facilitate a less obstructed view of these various embodiments of the present disclosure.

### DETAILED DESCRIPTION

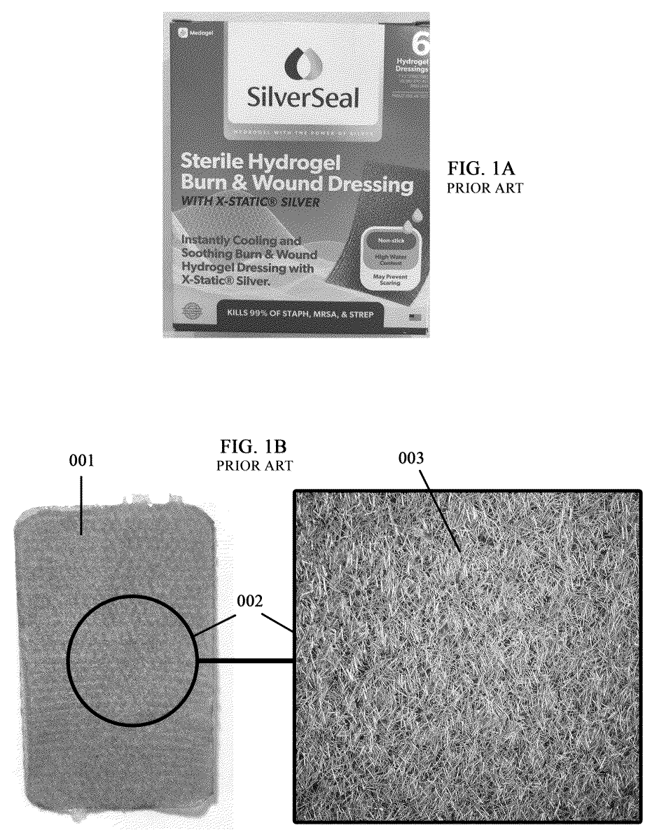

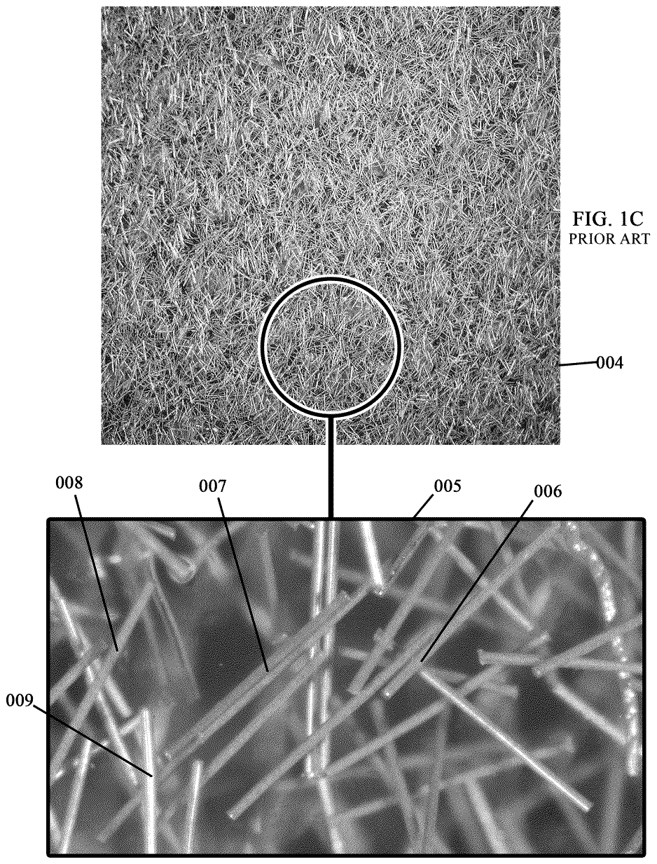

Image FIG. 1A shows prior art of SilverSeal burn and wound dressing with X-Static Silver. The product is a wound dressing with hydrogel with silver coated fibers to help with cooling and soothing burns. FIG. 1B is an image of prior art where 001 shows the SilverSeal product outside of the box and wrapping. 002 illustrates a zoomed image of the SilverSeal product. 003 illustrates the non-coated fibers and the silver coated fibers implemented into the hydrogel. FIG. 1C is an image of prior art SilverSeal, where 004 is a zoomed image of the silver coated fibers implemented into the hydrogel. 005 is a magnified view of the SilverSeal product showing a closer view of the silver coated fibers implemented into the hydrogel. 006, 007, 008 & 009 illustrate the coated fibers. The SilverSeal prior is a medical dressing, which is meant to maintain a sterile, moist environment and cool burnt wounds. In this case SilverSeal is a dressing meant to keep a moist environment on a burn wound using hydrogel and silver coated fibers implemented into the hydrogel. Even though SilverSeal has coated fibers and is meant for wounds, one in the skill trade would know that this prior art does not have a backing sheet associated with the structure of the dressing. Nor does it have a backing sheet with coated silver threads.

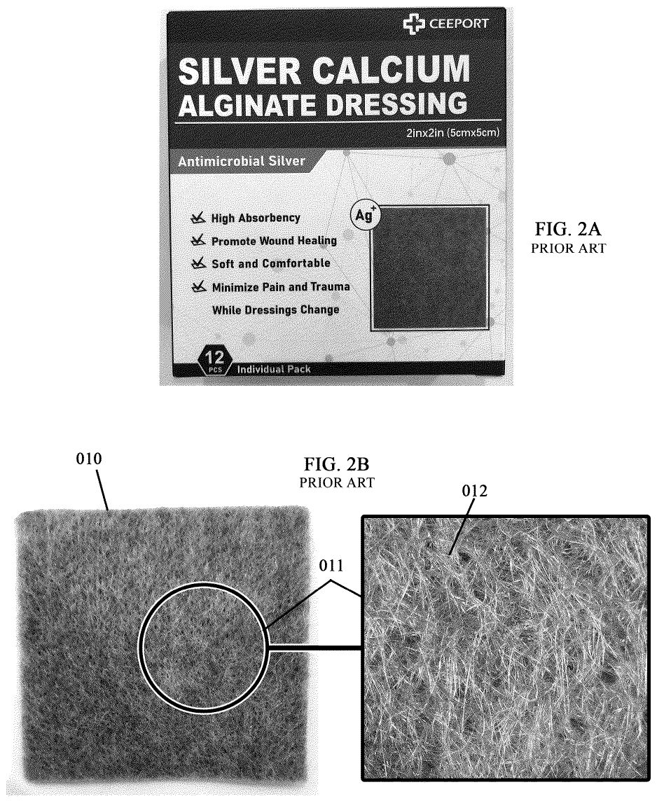

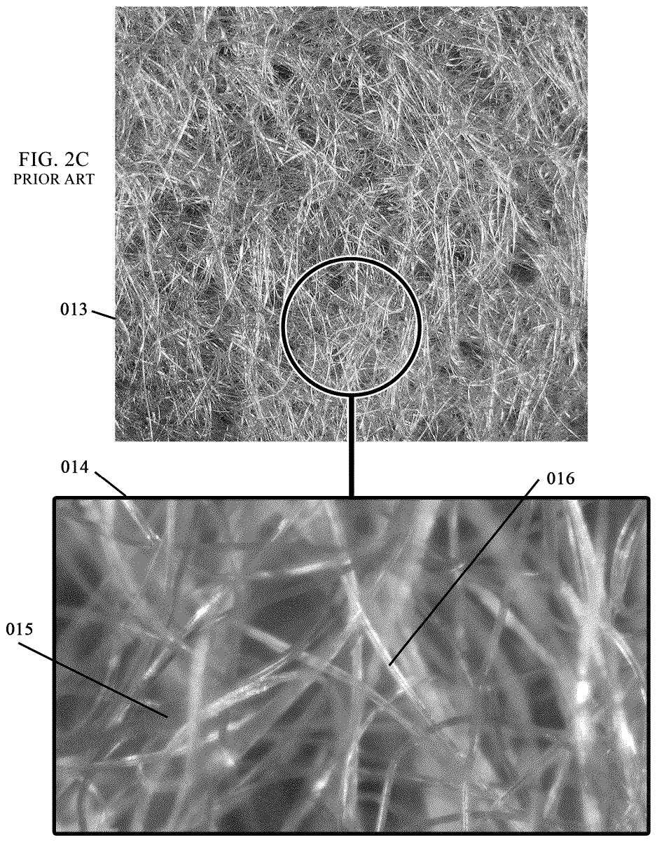

FIG. 2A is an image of prior are Silver Calcium Alginate Dressing by Ceeport. A high absorbent dressing with silver fibers and non-silver fibers implemented in the dressing. FIG. 2B is an image of prior art where 010 shows the Silver Calcium Alginate Dressing product out of the box and wrapping. 011 illustrates a zoomed image of the Silver Calcium Alginate product. 012 illustrates the silver fibers and non-silver fibers implemented in the dressing. FIG. 2C is an image of prior art Silver Calcium Alginate Dressing, where 013 is a zoomed image of the silver fibers and non-silver fibers implemented in the dressing. 014 is a magnified view of the Silver Calcium Alginate Dressing product showing a closer view of the silver fibers and non-silver fibers implemented in the dressing. 015 illustrates the coated fibers and 016 illustrates the non-coated fibers.

The Silver Calcium Alginate Dressing prior art is a medical dressing, which is meant to maintain a sterile, moist environment and absorb fluids. In this case Silver Calcium Alginate Dressing is a dressing meant to have high absorption properties with silver in it. Even though Silver Calcium Alginate Dressing has coated fibers and is meant for wounds. One in the skill trade would know that this prior art does not have a backing sheet associated with the structure of the dressing. Nor does it have a backing sheet with coated silver threads. These silver fibers are coated with a thin layer mixed with non-silver fibers give this dressing a low conductivity rate and using this dressing on a smart device will not work.

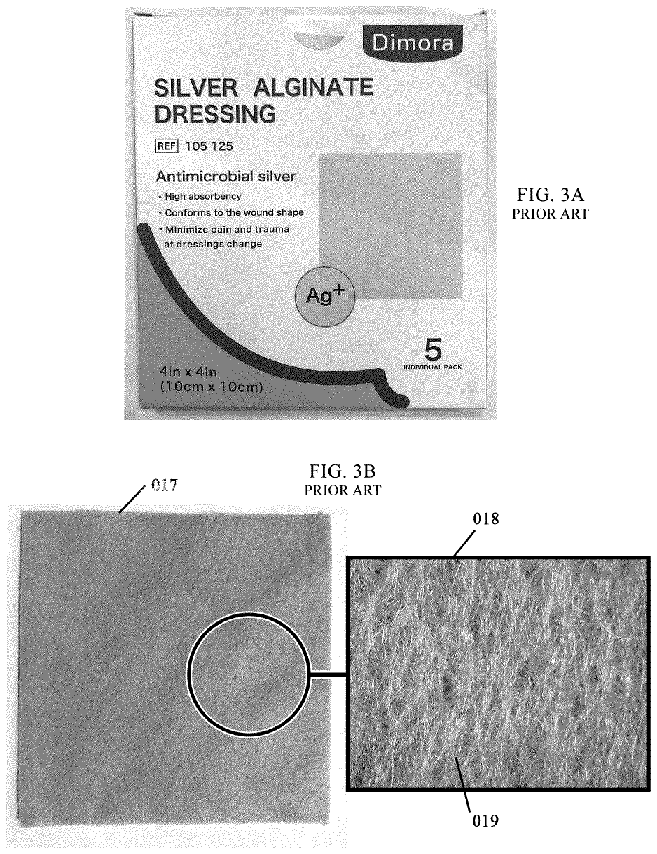

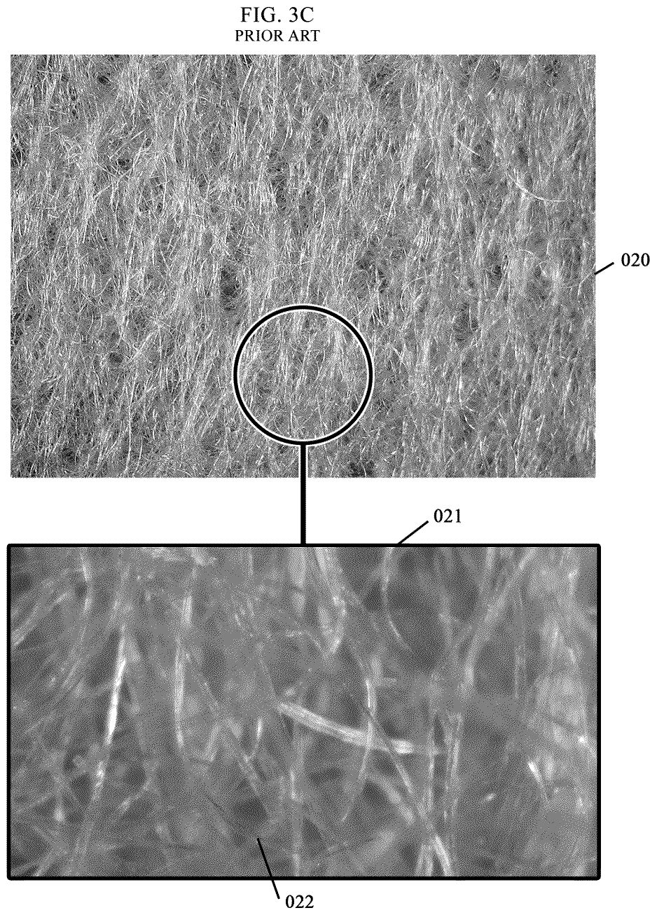

FIG. 3A is an image of prior are Silver Alginate Dressing by Dimora. A high absorbent dressing with silver nano particles is implemented in the dressing. FIG. 3B is an image of prior art where 017 shows the Silver Alginate Dressing product out of the box and wrapping. 018 illustrates a zoomed image of the Silver Alginate Dressing product. 019 illustrates the silver nano particles implemented in the dressing.

FIG. 3C is an image of prior art Silver Calcium Alginate Dressing, where 020 is a zoomed image of the silver nano particles implemented in the dressing. 021 is a magnified view of the Silver Alginate Dressing product showing a closer view of the silver nano particles implemented in the dressing. 022 illustrates the silver nano particles implemented in the dressing. The Silver Alginate Dressing prior art is a medical dressing, which is meant to maintain a sterile, moist environment and absorb fluids. In this case Silver Alginate Dressing is a dressing meant to have high absorption properties with silver in it. Even though Silver Alginate Dressing has silver nano particles implemented in the dressing and meant for wounds. One in the skill trade would know that this prior art does not have a backing sheet associated with the structure of the dressing. Nor does it have a backing sheet with coated silver threads. These silver nano particles implemented in the dressing also have an exceptionally low conductivity rate and using this dressing on a smart device will not work.

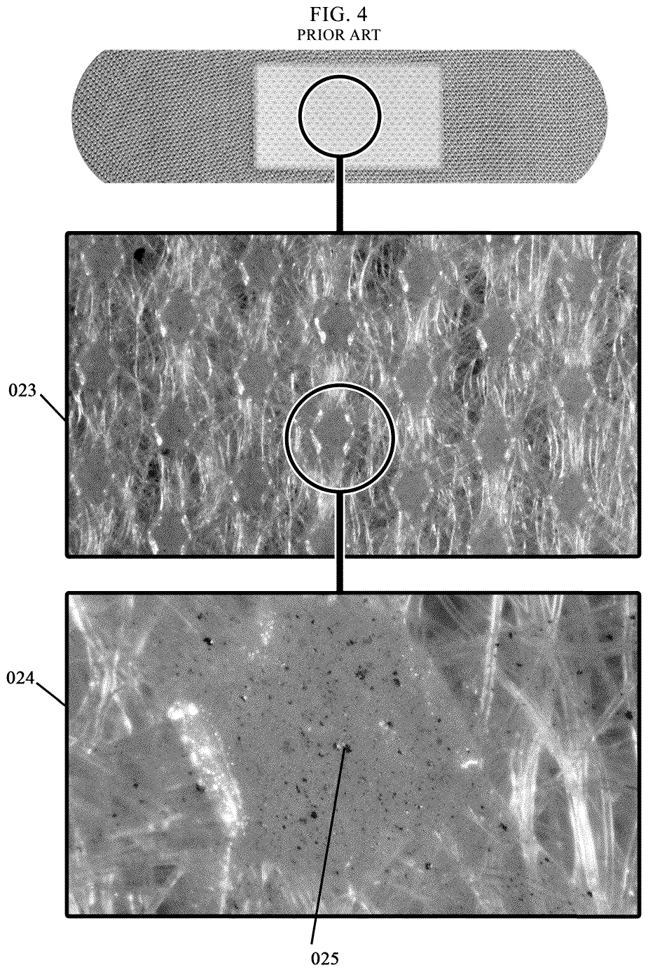

FIG. 4. Illustrates an image of prior art of an adhesive bandage with nano-silver flakes on the absorption pad. 023 illustrates a zoomed view of the nano-silver flakes on the absorption pad. 024 illustrates a magnified view of the nano-silver flakes implemented on the absorption pad. 025 illustrates the nano-silver flakes implemented on the absorption pad. Even though this prior art is an adhesive bandage with nano-silver flakes on the absorption pad. The backing sheet associated with the prior art does not have silver coated threads on it. These silver nano particle flakes implemented in the absorption pad also have an exceptionally low conductivity rate and using this absorption pad on a smart device will not work.

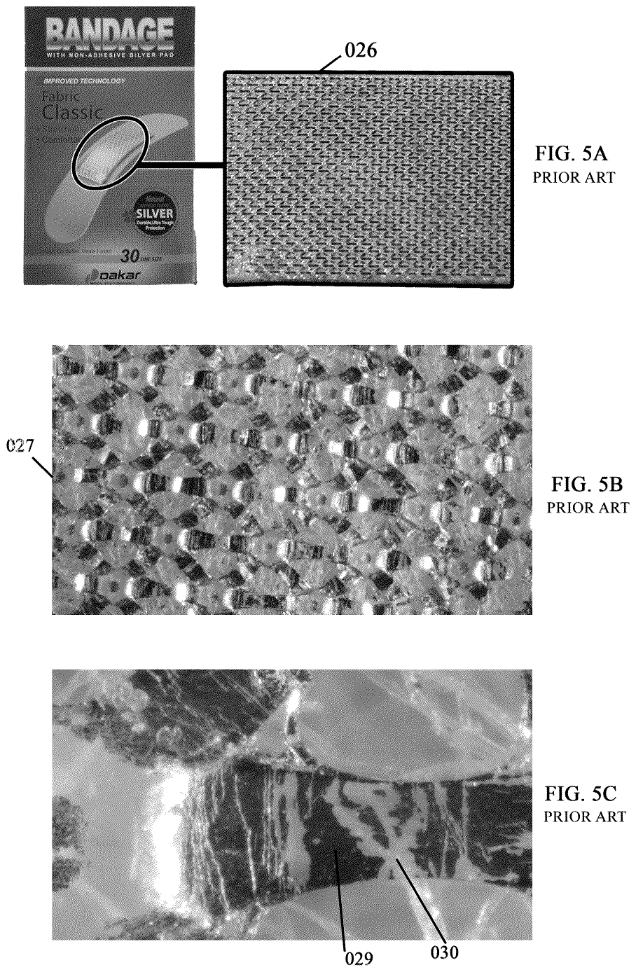

FIG. 5A Illustrates prior art of an adhesive bandage by Daker with a silver film on an absorbent pad. 026 illustrates the prior art image of the silver absorbent pad. 027 illustrates a zoomed-up view of the prior art image of the silver absorbent pad. 028 illustrates a magnified view of the silver absorbent pad. 029 illustrates the silver film associated with the absorbent pad and 030 illustrates the breakage point where the silver film flaked off polymer layer of the absorbent pad. The backing sheet associated with this prior art does not have silver coated threads on it. This prior art illustrates a silver film on the absorbent pad which is structurally different than a backing sheet. Absorbent pads have been made in a way to absorb fluids. Even though backing sheets and absorbent pads can both be made from cotton. The absorbent pads material is made thicker, softer, and the material is expanded with a fluffier texture to it. These features give the absorbent pad the ability to absorb substantial amounts of fluid over the backing sheet layer. While the backing sheet layer does the opposite and is made not to absorb large amounts of fluid. Even though some backing sheets are made with cotton and might absorb some fluids it is significantly less than an absorbent pad. The structure of a backing sheet is less dense, material is compressed and does not have that fluffy texture to it.

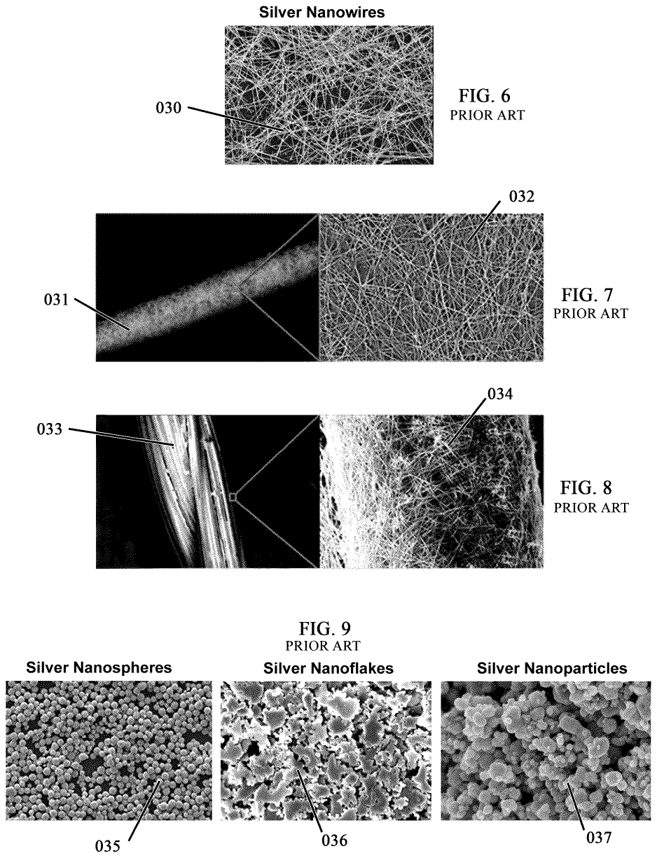

FIG. 6 Image illustrates prior art of silver nanowires coatings and 030 illustrates the nano wires.

FIG. 7 Image illustrates prior art of silver nanowire coating on a thread. 031 illustrates the thread and 032 is a zoomed view of the silver nanowires on the thread. The image shows a coating with a thickness of around 0.02 microns. Coatings around 0.02 microns have lower conductivity and antimicrobial properties.

FIG. 8 Image illustrates prior art of a silver nanowires coating with a PET coating on the thread. 033 illustrates the thread and 034 is a zoomed view of the silver nanowires on the thread. The image shows a coating with a thickness of around 0.02 microns. Coatings around 0.02 microns have lower conductivity and antimicrobial properties.

FIG. 9 Image illustrates prior art of various silver nano-particle geometries. 035 illustrates silver nanospheres, 036 illustrates silver nanoflakes, and 037 silver nanoparticles and nanoflakes. A backing sheet in accordance with the present disclosure can combine various oligodynamic nano coating techniques like silver. These nanocoating's can be combined with various oligodynamic materials like silver, copper, brass and gold. In some embodiments the backing sheet can have a combination of oligodynamic materials on it with a combination of various nanoparticle geometries.

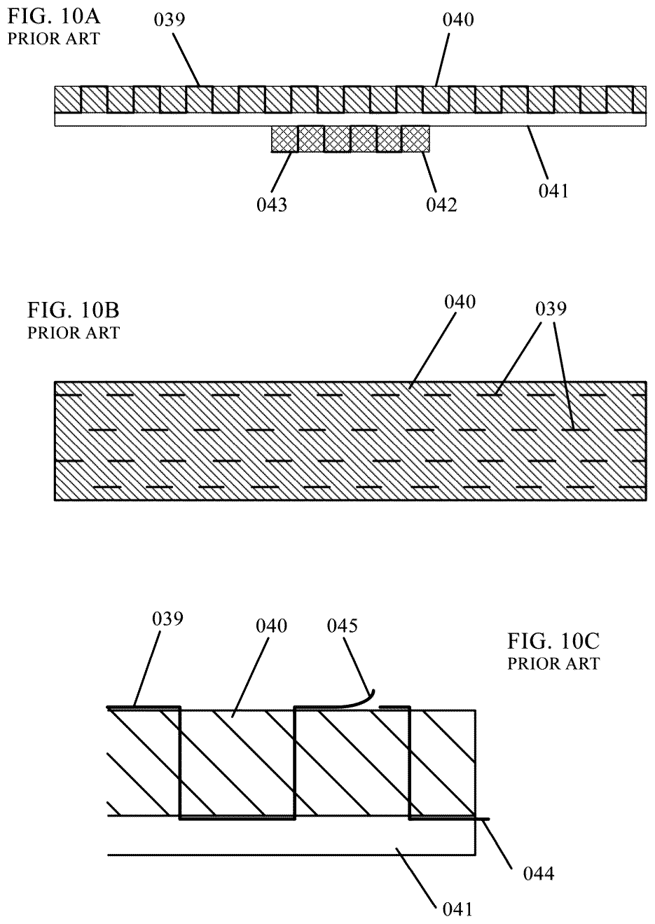

FIGS. 10A, 10B and 10C illustrates prior art of adhesive bandage with metallic threads implemented on an existing fabric bandage. While the implemented weave patterns provided by metallic strands 039 and/or 043 would provide effects of having metallic material close to the skin and/or wound of a user, the implemented weave pattern provided by metallic strands 039 and 043 can be problematic, either near sensitive skin of a user or near an open wound site of a user. FIG. 10C illustrates the bandage of FIG. 10A in magnified detail, showing the potential drawbacks of a metallic strand implemented weave pattern. Cloth layer 040 and adhesive layer 041 are illustrated in a side view. Metallic strand 039 is illustrated being woven through cloth layer 040. Metallic strand 039 is exposed on an external surface of cloth layer 040, leaving metallic strand 039 open to being snagged or caught upon foreign objects. Further, metallic strand 039, if it is made substantially of metal, is subject to snapping if bent repeatedly or at too sharp of an angle. An end 044 of metallic strand 039 is illustrated poking outwardly from an end of cloth layer 040, and a snapped end 045 of metallic strand 039 is illustrated poking upwardly from cloth layer 040. It will be appreciated that snapped end 045 and similar snapped ends can occur anywhere along a length of metallic strand 039 and can project in any direction from cloth layer 040. As a result, either end 044 and snapped end 045 can be an irritant, poking and scratching the skin and/or wound of the user.

11A & 11B illustrates an image of an oligodynamic coating on with the threads cut in half showing coating thickness. 039A illustrates the oligodynamic coating on the thread, 039B illustrates the non-coated threads and 039C illustrates the oligodynamic coating thickness dimension variance. 039D illustrates a group of oligodynamic coating on the thread cut in half. The following embodiment coating thickness can range from less than 0.5 microns, 0.5-3 microns, 1-6 microns, 0.02-6 microns or less, and combination or variety of the following. A coating thickness of around 1 micron on the thread gives a backing sheet durability, less breakage, higher antimicrobial properties, and conductibility in accordance with the present disclosure.

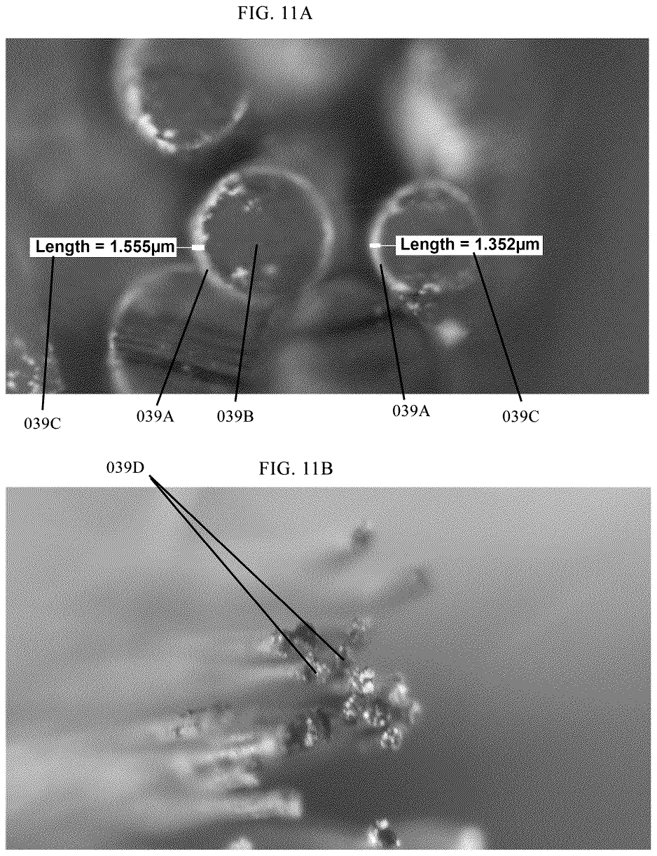

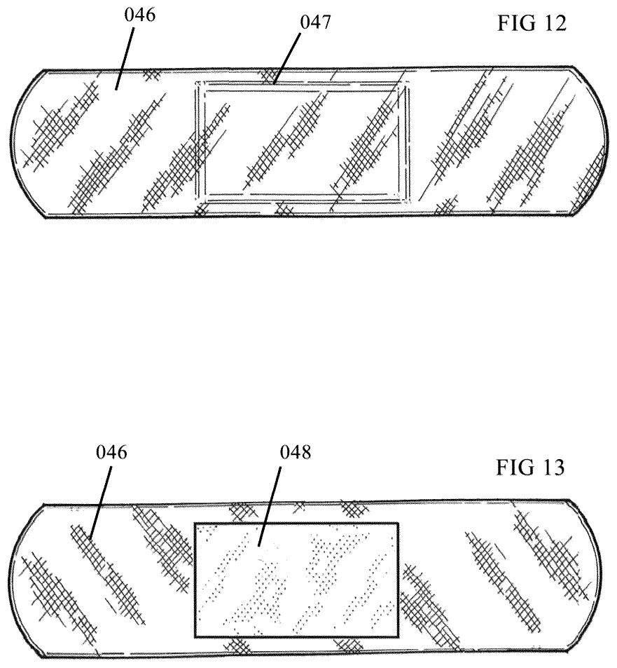

FIG. 12. Illustrates a top view sketch of an oligodynamic backing sheet. 046 illustrates the oligodynamic backing sheet cloth fabric. 047 illustrates an indent of an island style absorbent pad located under the cloth layer located at the center of the backing sheet.

FIG. 13. Illustrates a bottom view sketch of an oligodynamic backing sheet. 046 illustrates the oligodynamic backing sheet cloth fabric. 048 illustrates an island type absorbent pad located at the center of the backing sheet.

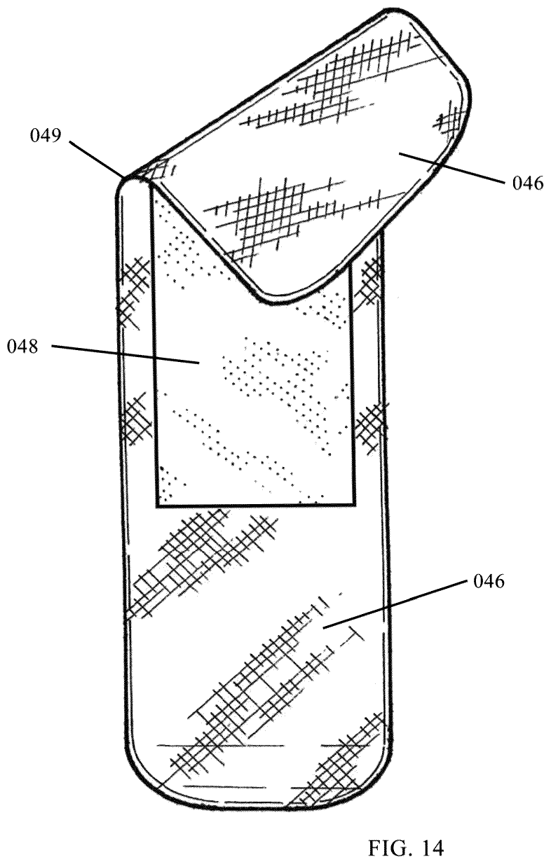

FIG. 14. Illustrates a bottom view sketch of an oligodynamic backing sheet. 046 illustrates the oligodynamic backing sheet cloth fabric. 048 illustrates an absorbent pad located at the center of the backing sheet. 049 illustrates bending of the oligodynamic backing sheet fabric showing its flexibility

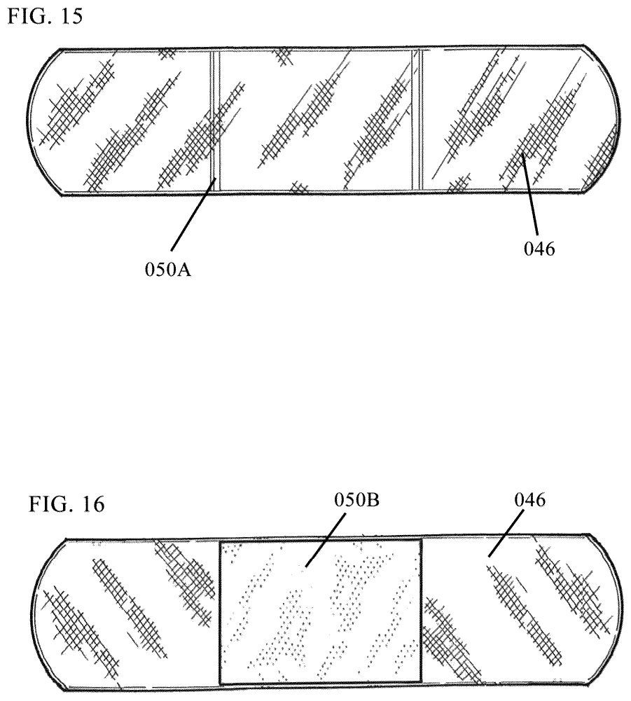

FIG. 15. Illustrates a top view sketch of an oligodynamic backing sheet. 046 illustrates the oligodynamic backing sheet cloth fabric. 050A illustrates an indent outline of the absorbent pad under the cloth layer located at the center of the backing sheet that extends to edge of the bandage.

FIG. 16. Illustrates a bottom view sketch of an oligodynamic backing sheet. 046 illustrates the oligodynamic backing sheet cloth fabric. 050B illustrates the absorbent pad located at the center of the backing sheet that extends to edge of the bandage.

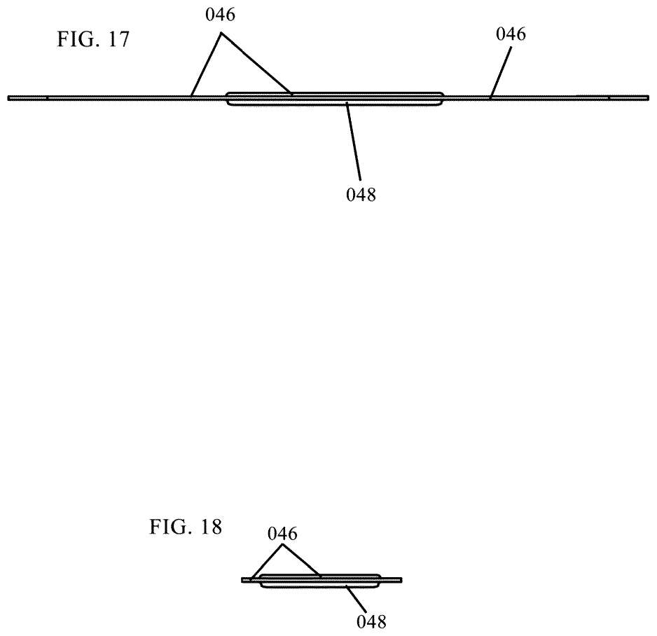

FIG. 17. Illustrates a side view sketch of an oligodynamic backing sheet. 046 illustrates the oligodynamic backing sheet cloth fabric. 048 illustrates the absorbent pad located at the center of the backing sheet.

FIG. 18. Illustrates a front view sketch of an oligodynamic backing sheet. 046 illustrates the oligodynamic backing sheet cloth fabric. 048 illustrates the absorbent pad located at the center of the backing sheet.

FIG. 19. Illustrates a top view sketch of an oligodynamic backing sheet. 046 illustrates the oligodynamic backing sheet cloth fabric. 051 illustrates a zoom view of the oligodynamic backing sheet.

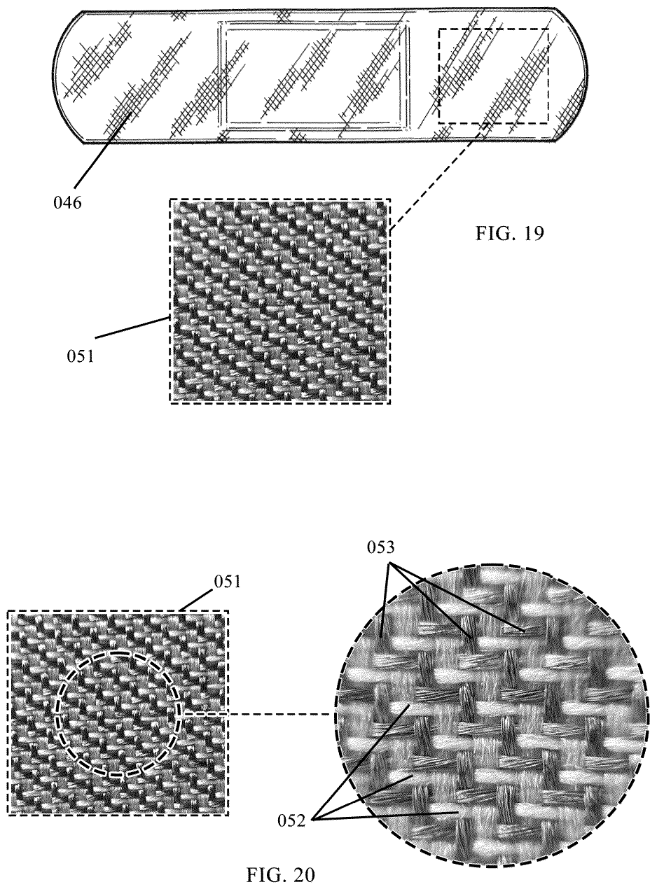

FIG. 20. Illustrates a magnified view of the oligodynamic backing sheet. 051 is a magnified view of the oligodynamic coated threads. 052 is magnified view of threads without any oligodynamic coating. 053 illustrates oligodynamic coated threads. The coated threads of 053 can be a combination or multiple combinations of various oligodynamic coated threads. For example, coated silver threads with copper coated threads. 053 can also be just a single type of coated thread, for example silver threads. The oligodynamic coated threads of 053 can be coated over various materials. Materials such as cotton, polyester, bamboo, hemp, polymers, latex, rubber and other common materials associated with bandage manufacturing can be coated with oligodynamic material like silver and not limited to silver. 052 non-coated threads can be made from materials such as cotton, nylon, polyester, bamboo, hemp, polymers, latex, rubber and other common materials associated with bandage manufacturing. The coated and non-coated threads that make the backing sheet can be stitched together in various ways. Some examples are and not limited to check, hexagonal, braided, twined, wrapped wicker, plaiting, coiling, cycloid, crossed, weaved, knit, twill and other standard manufacturing stitching processes. The backing sheet is not limited to a combination of these stitching processes. The non-coated threads can be manufactured in various colors. The coated threads can be manufactured in various colors. The non-coated threads can be manufactured with a water repellent agent. The coated threads can be manufactured with a water repellent agent. The entire oligodynamic backing sheet can be manufactured with a water repellent agent.

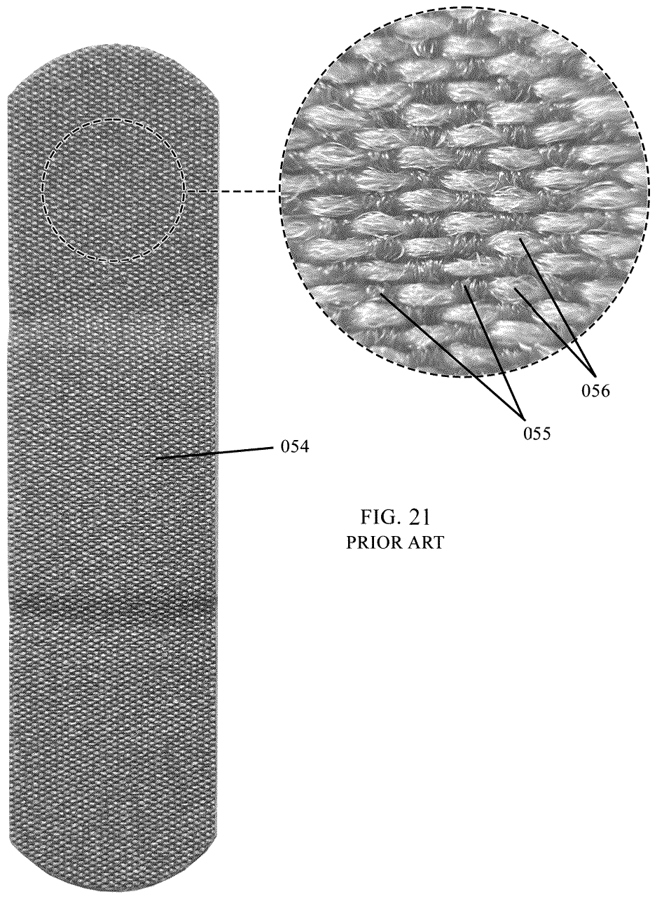

FIG. 21. Illustrates prior art of a standard fabric adhesive bandage backing sheet. 054 illustrates how the prior art does not have any oligodynamic coated threads associated with the backing sheet. 055 illustrates a magnified view of the backing sheet material with a cotton thread with a darker color appearance to it. 056 illustrates a magnified view of the backing sheet material with a cotton thread with a lighter color appearance to it. Both 055 and 056 do not contain any oligodynamic coating on the threads.

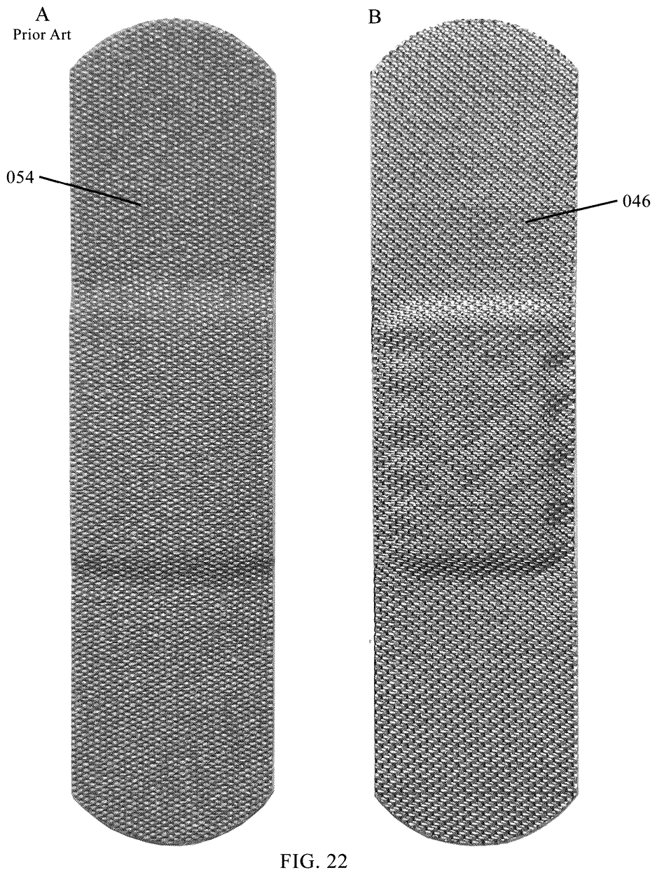

FIG. 22. Label A Illustrates prior art of a standard fabric adhesive bandage backing sheet without any coated threads next to an oligodynamic coated thread backing sheet adhesive bandage labeled B. 054 illustrates the non-coated cotton threads on the backing sheet. 046 illustrates the silver coated threads on the backing sheet.

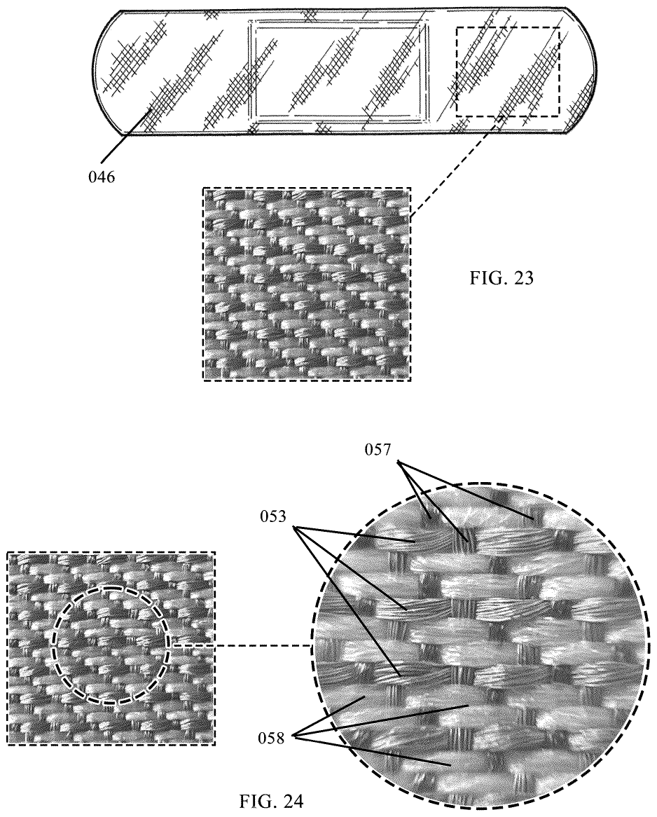

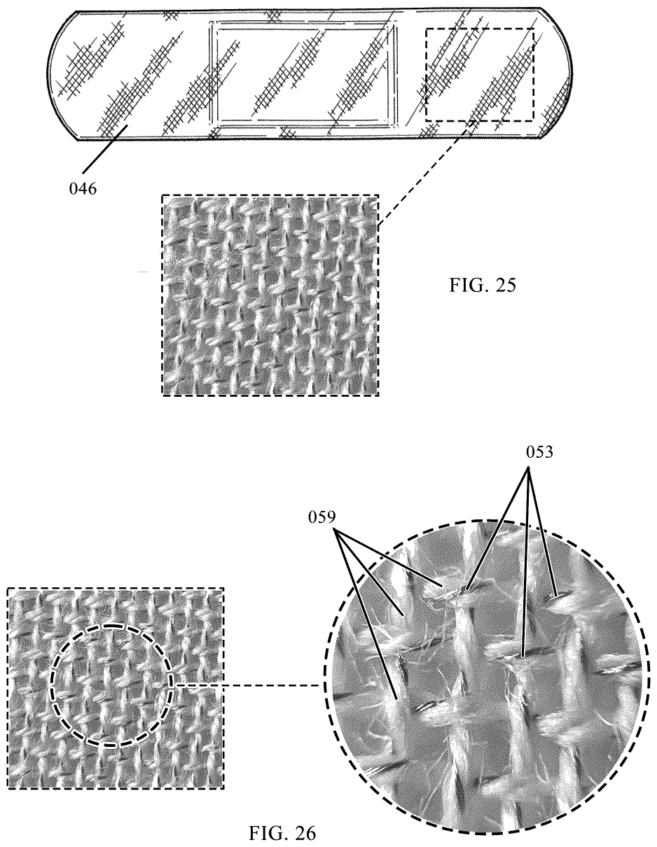

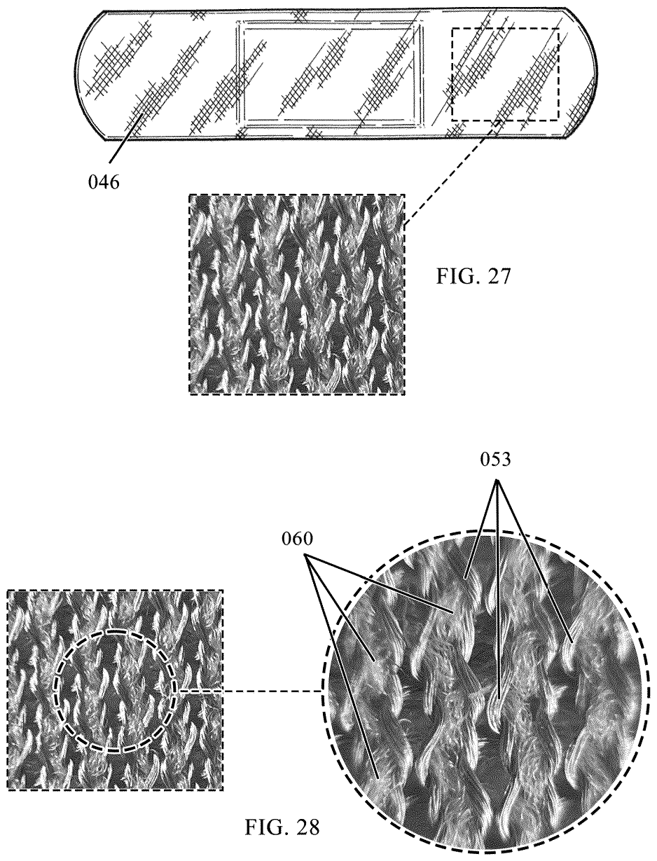

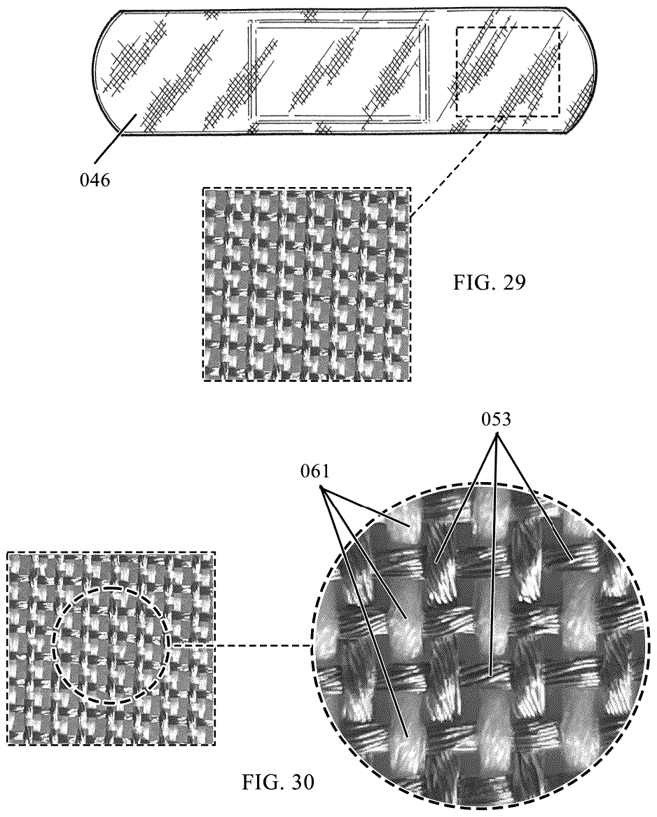

FIG. 23-30 Illustrates various oligodynamic coated threads patterns on a backing sheet. 046 illustrated the backing sheet and 053 illustrates the oligodynamic coated threads. 057-061 illustrates the non-coated oligodynamic threads. FIG. 23-30 also Illustrates the magnified views of the oligodynamic coated threads on the backing sheet and that these coated threads can be in various thread widths and patterns when manufactured and with the non-coated threads.

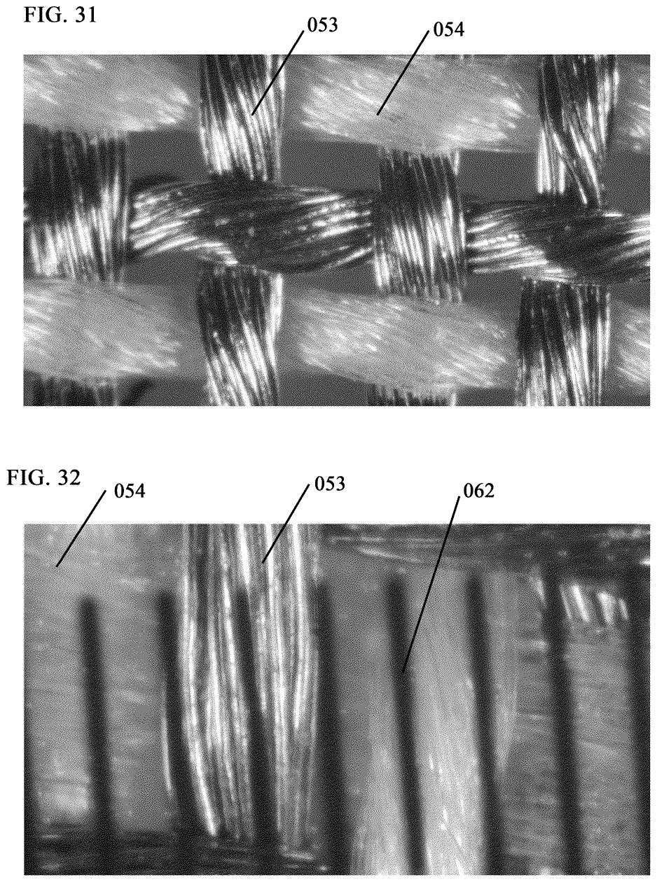

FIG. 31-32. illustrates an image of a magnified view of the oligodynamic coating of the threads on the backing sheet. 053 illustrates the oligodynamic coated threads and 054 illustrates the non-coated threads. 062 illustrates a ruler over the backing sheet to visualize the coated thread thickness. Each individual line on the rule represents. 1 mm. The oligodynamic coating on the threads or strain can have a width of 0.05 mm-0.6 mm, 0.05 mm-1 mm, and 0.01 mm-1 mm. larger widths will increase cost and are meant for longer usage. For class 1-2 medical devices for under 48 hours of usage are ideal to provide an affordable and effective bandage.

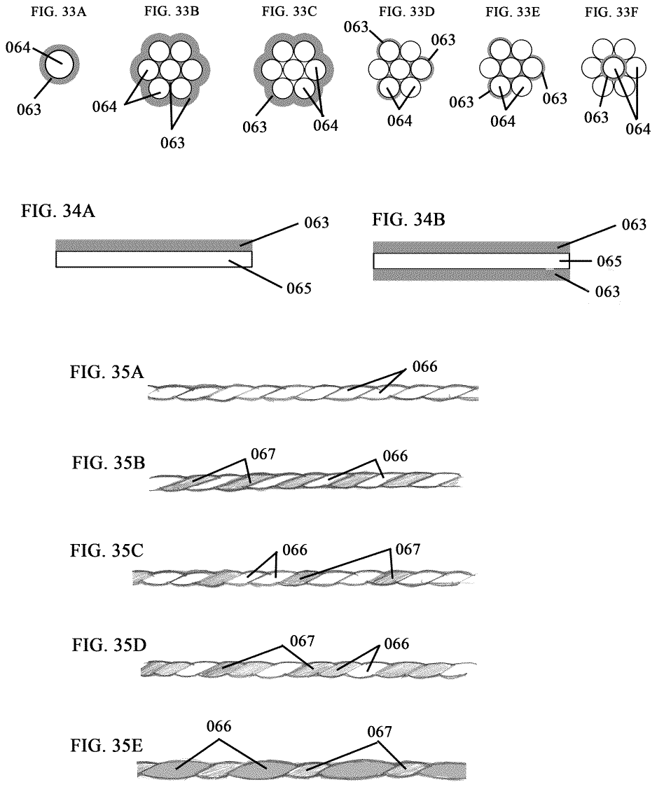

FIG. 33A-33G illustrates in magnified view of an oligodynamic coated threads, bundle of strands or fibers coated with an oligodynamic coating. 063 illustrates the oligodynamic coating and 064 illustrates the non-coated materials. The oligodynamic coating can be on single strain, fiber or thread made in a bundle with other oligodynamic strain, fiber or thread. Thread can be made of one or more coated strains, fibers or threads and combined with non-coated strains, fibers or threads. These strains, fibers or threads can be placed in various combinations when making the thread bundle. Various oligodynamic coated threads like silver, copper, or gold but not limited to other oligodynamic materials can be combined, mixed/matched to make the bundle thread.

FIG. 34A. Illustrates an oligodynamic backing sheet with the top layer has a coating on it. Where 065 illustrates a non-coated backing sheet layer and 063 illustrates the oligodynamic material coated on top of the backing sheet.

FIG. 34B. Illustrates an oligodynamic backing sheet with the top and bottom layer has a coating on it. Where 065 illustrated a non-coated backing sheet layer and 063 illustrates the oligodynamic material coated on top and on the bottom of the backing sheet.

FIG. 35A-35E Illustrates various threads spun together with an oligodynamic coating on it. 066 Illustrates a non-coated thread and 067 Illustrates an oligodynamic coated thread. These threads can be spun, braided or stitched together to create a thread. The full thread can be coated with oligodynamic materials. Thread can be a combination of multiple types of oligodynamic materials spun, braided or stitched together. The thread can be a combination of oligodynamic coated thread and non-coated thread. Thread can be a combination of multiple types of oligodynamic coated threads with non-coated threads.

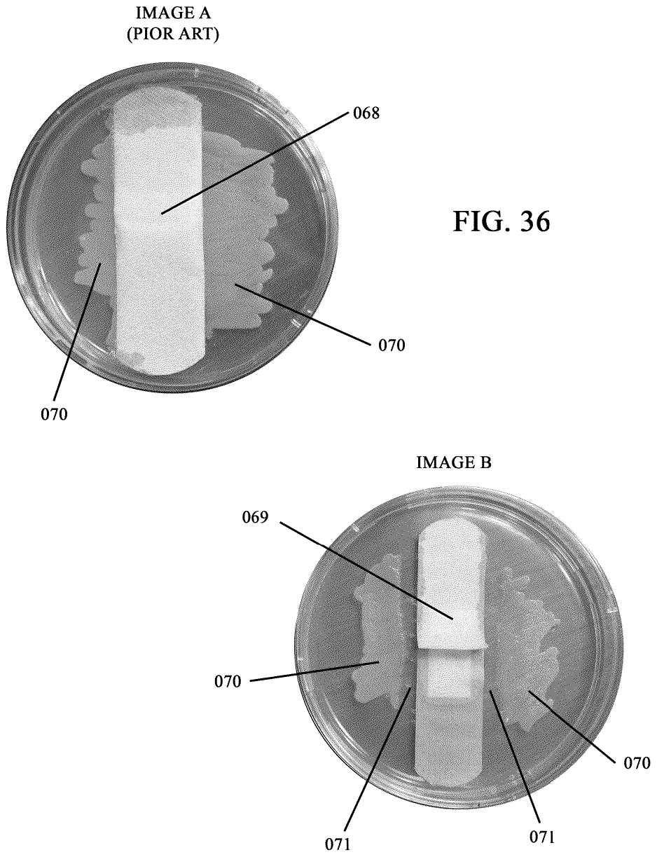

FIG. 36. Image A illustrates prior art of standard adhesive bandage without any oligodynamic coated threads on the fabric backing sheet in a petri dish. 068 illustrates the location of prior art in petri dish. Image B illustrates the present disclosure of an adhesive bandage with coated oligodynamic threads on the fabric backing sheet in a petri dish. 069 illustrates the location of present disclosure art in petri dish. 070 illustrates bacteria growth in a 24-hour period. 071 illustrates portion without bacteria growth. The antimicrobial properties of the oligodynamic coated threads on a backing sheet created a germ barrier to protect the wound from germs. While prior art does not have this effect on germs due to the fact it does not have oligodynamic coated threads on it.

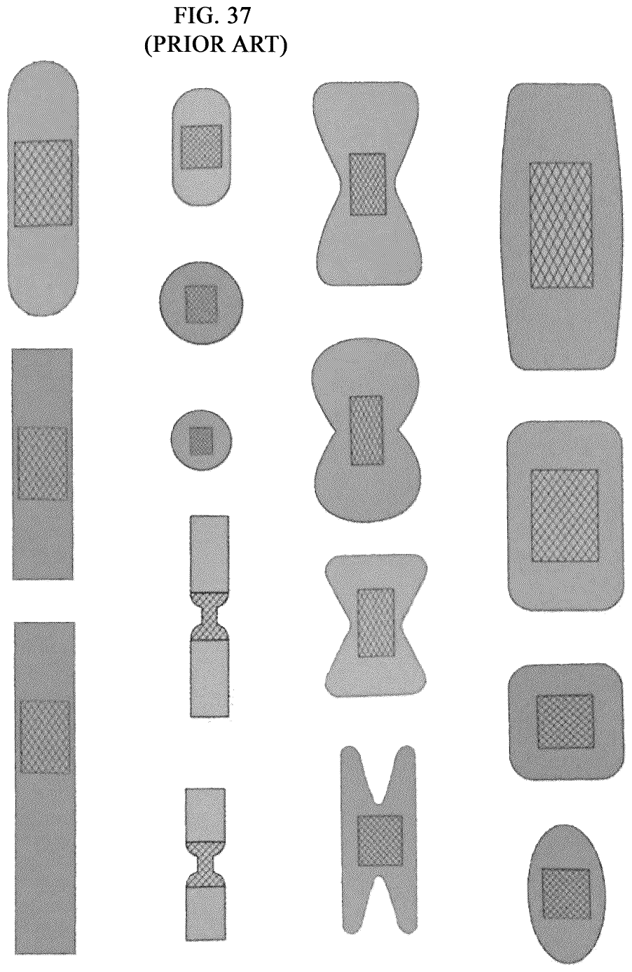

FIG. 37. Shows prior art of various adhesive bandage shapes from strip, patch, square, rectangular, fingertip, spot, knuckle, wraparounds, butterfly, anchor, finger, elbow, heels, extension, and not limited to other shapes in the adhesive bandage medical field. That all these various bandage shapes, sizes, and styles can have an oligodynamic coated backing sheet on them.

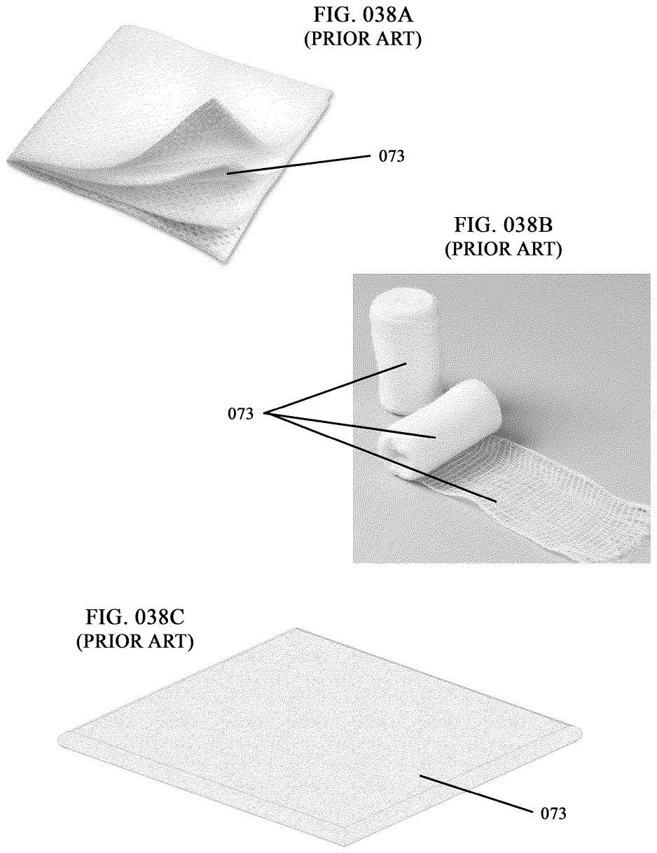

FIGS. 38A, 38B, & 38C illustrate prior of a gauze pad. These gauze pads can be made in single sheets, or in a roll form. 073 illustrates how gauze pads are made to have absorbing properties and materials meant to absorb fluids from the wound. The prior art gauze examples do not have backing sheets on them. The absorbing properties of 073 can be manufactured by a thick dense material as in FIG. 38C or in a multilayer mesh formation as in FIGS. 38A and 38B.

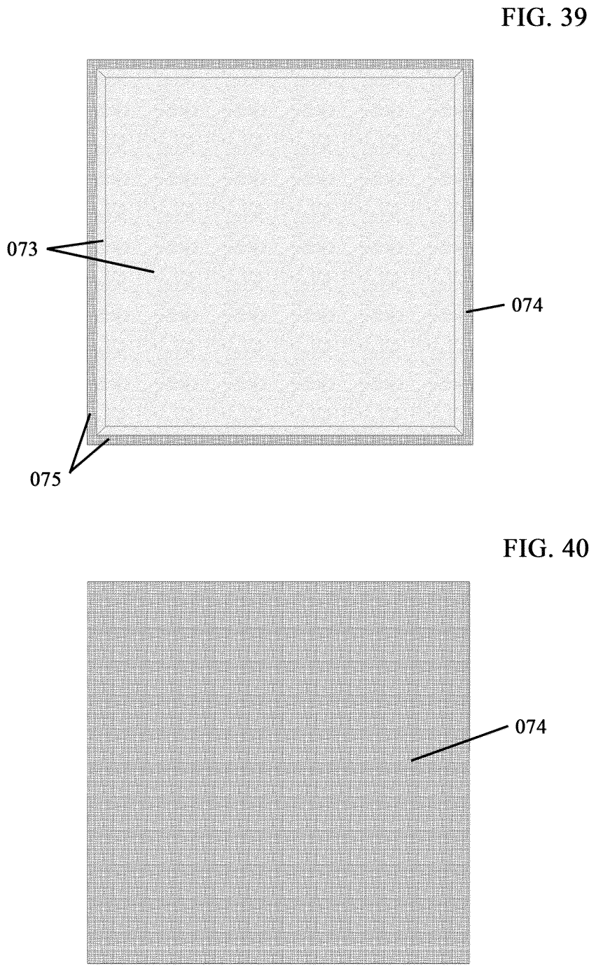

FIGS. 39 & 40 illustrate a square medical gauze pad with an oligodynamic coated backing sheet in accordance with the present disclosure. The embodiment mentioned in this application is not limited to a square gauze structure but can be made into various shapes like round, rectangular, triangle, hexagonal etc. They can also be manufactured into single sheets or into rolls. 074 shows the backing sheet with oligodynamic coating on it. 075 shows a periphery of the oligodynamic backing sheet around the gauze pad 073. The periphery can have adhesive or no adhesive on it. These embodiments mentioned in this application can be made with a periphery or without a periphery. The periphery on the oligodynamic backing sheet is meant to contain the fluids from the absorbent material of the gauze pad from spilling out on the sides.

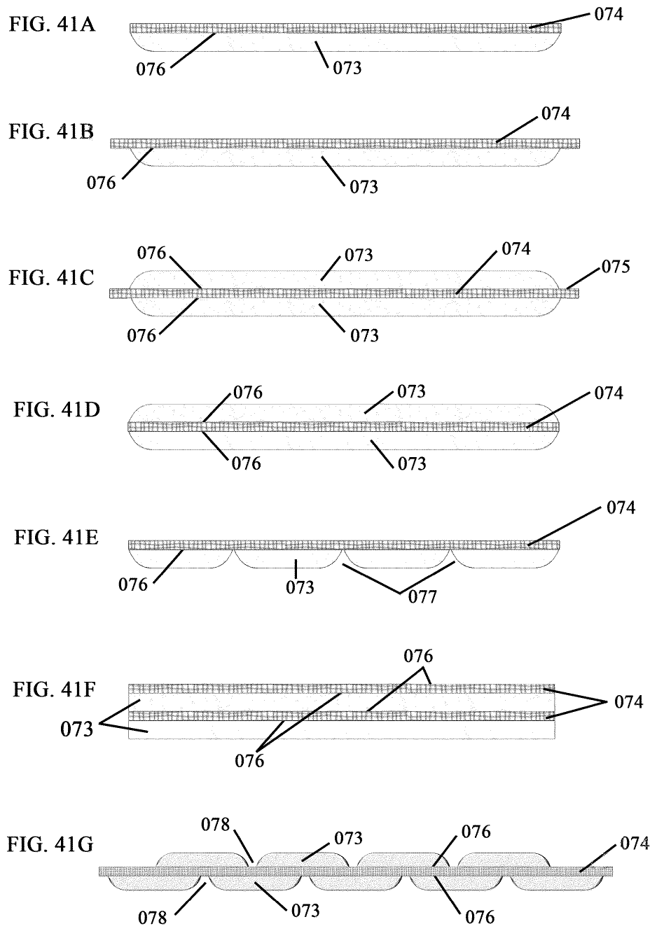

FIGS. 41A-F41F. illustrates side views of a medical gauze pad with an oligodynamic coated backing sheet with a gauze pad associated with the backing sheet. 074 shows the oligodynamic coating backing sheet, 075 shows a periphery of the oligodynamic backing sheet. 073 illustrates the gauze pad. 076 illustrates how the oligodynamic backing sheet and gauze pad can be joined by an adhesive or sewed together. 075 periphery can be manufactured with or without an adhesive on the periphery portion of it. 077 illustrates that the gauze pad can be made into various sections and spacing is not limited to one particular structure. FIG. 41F illustrates a stackable structure of gauze pad and backing sheet. The oligodynamic backing sheet can also have a perforated structure to it. Since the backing sheet is not meant for absorption, this perforation on the backing sheet can be used to distribute blood flow. In some cases, fluids will build up more on certain areas of the gauze pad over other areas. It all depends on placement of the gauze and amount of fluids flowing out of the wound. These perforations can distribute fluids in other areas of the gauze, so it is not all concentrated in one area. Which in return will prevent overflow of fluids seeping out of the bandage. FIG. 41G. illustrates 078 a gauze pad that is offset from the gauze pad located at the bottom. When the oligodynamic backing sheet has a perforation on it the access fluids could leak out from that perforation so an offset gauze pad can be placed over that perforation to stop fluid leakage. Gauze portion 073 can also be made in various shapes and sizes and not limited to one particular shape or size.

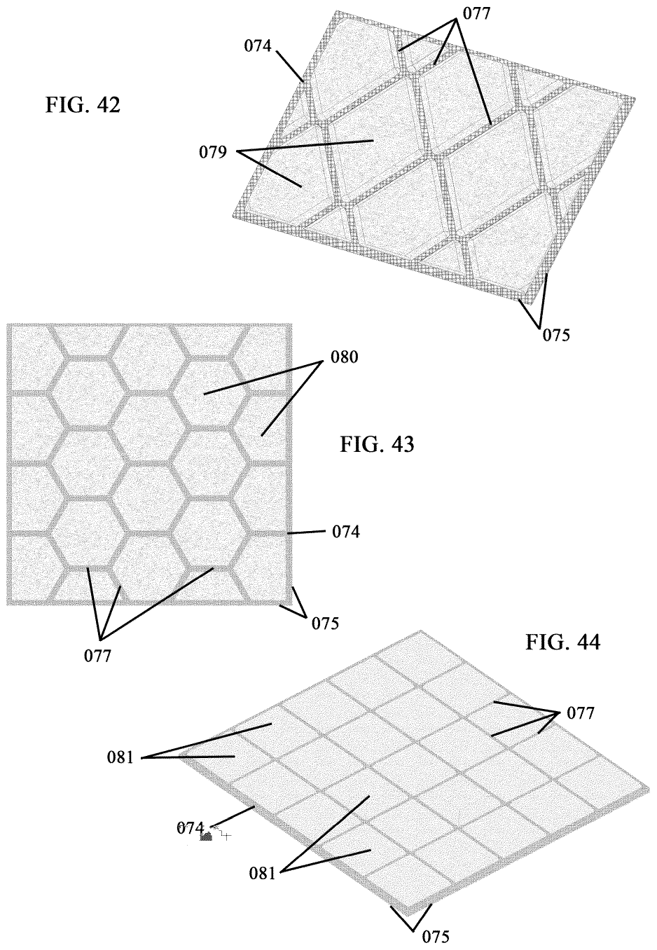

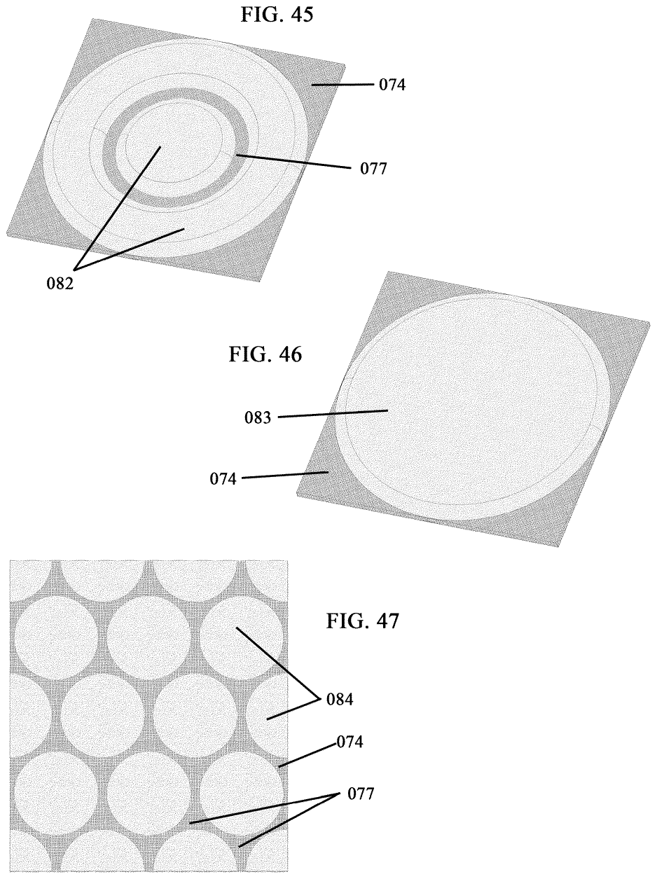

FIGS. 42-47. Illustrates a medical gauze pad with an oligodynamic coated backing sheet with various gauze patterns. 079 illustrates a gauze pad with a diamond shape to it. 079 also illustrates how these diamond shape gauze pads are scattered in multiple sections throughout the oligodynamic backing sheet. 077 illustrates spacing between the gauze pads. The following embodiments are not limited to a non-spaced section. 075 illustrates a periphery but not limited to non-periphery structures. The periphery can be used to stop fluids from spilling out from the edges. 080 illustrates gauze with a polygonal shape and 081 illustrates a square pattern. 082 illustrates a dual circle gauze pad, 083 illustrates a single circle gauze pad, 084 illustrates a multi circle gauze pad. Gauze pad can also be made in a spiral shape. 074 illustrates oligodynamic coated backing sheet. The backing sheet is not limited to a square design but can be in the form of a circle, rectangular, polygon, or odd shape patterns.

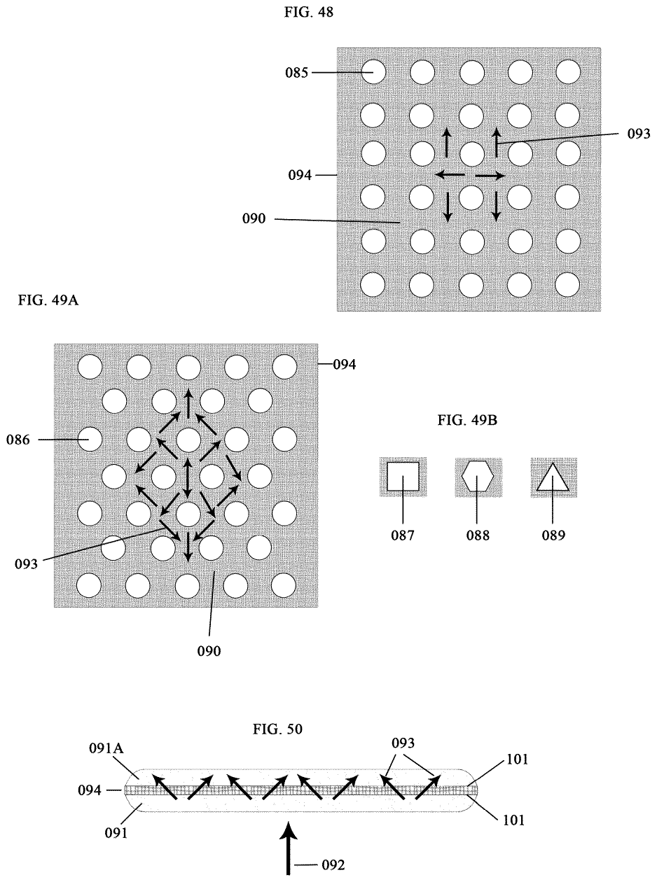

FIGS. 48-49 illustrate an oligodynamic backing sheet with a perforation structure. 094 illustrates the oligodynamic backing sheet with perforations on it. 085 illustrates circle perforation on the oligodynamic backing sheet in a uniform pattern. 086 illustrates circle perforation on the oligodynamic backing sheet in an offset pattern. 090 illustrates the spacing of the perforation on the oligodynamic backing sheet. 087 illustrates a square perforation, 088 illustrates a polygonal perforation, and 089 illustrates a triangle perforation. The following embodiments are not limited to other perforations such as rectangles, ovals, various polygonal shapes, and odd shapes. 090 illustrates the spacing of the perforations and not limited to a particular spacing structure. 093 illustrates the fluid distribution due to the perforation on the oligodynamic backing sheet. Since a backing sheet has little to no absorbance properties it can be manufactured in perforations to distribute fluids throughout the bandage.

FIG. 50. Illustrates the distribution of fluids from a wound due to the perforation on the oligodynamic backing sheet. 091 illustrates a gauze pad, 094 illustrates the oligodynamic backing sheet with perforations on it. The backing shape is illustrated in a square structure but is not limited to others such as a circle, polygon rectangular etc. 092 is the fluid direction from a wound, 093 is the fluid distribution due to the perforation on the oligodynamic backing sheet. The structure of the oligodynamic backing sheet with perforations makes the fluid pass through the antimicrobial properties of the backing sheet and distribute fluid evenly throughout the bandage. These features make the gauze bandage spill out less fluids in certain areas of the bandage and provide antimicrobial protection at the same time. These perforations can be 1 mm in diameter or larger, 1 mm-3 mm, 2-6 mm, 2-15 mm, 1-15 mm, and 1-18 mm. Not limited to other sizes if the perforations are equal or larger than the porous or mesh structure of some absorbent materials that gauze pads can be made from.

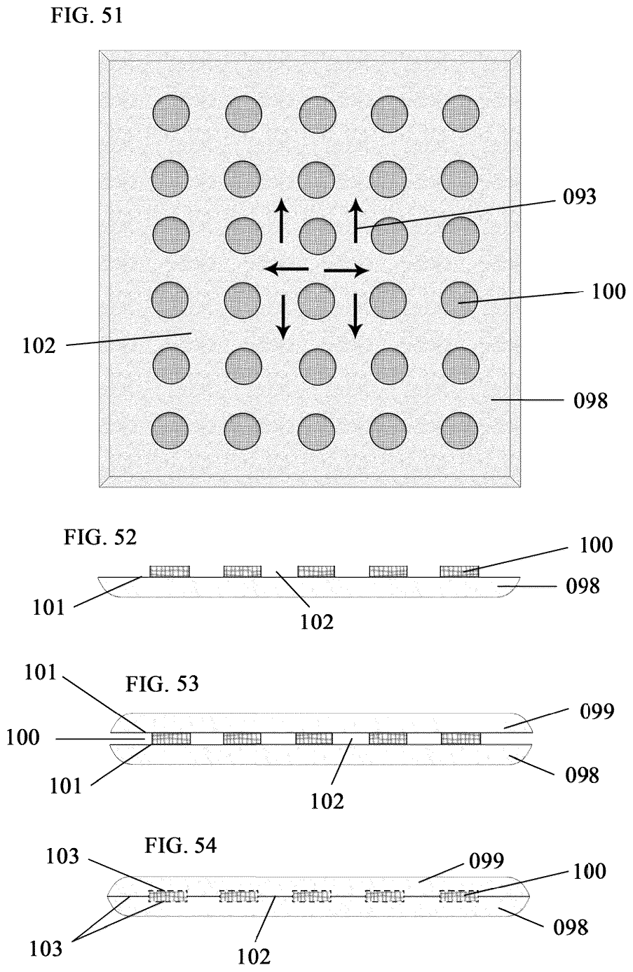

FIGS. 51-54 Illustrates a gauze pad with oligodynamic backing sheet segmented structure shapes on it. 100 illustrates circle shape of the backing sheet segments. These shapes are not limited to other shapes such as ovals, squares, rectangles, polygonal, triangles or any shape or odd shape structure. Also not limited to a combination of various shapes. 093 is the distribution of direction of fluid due to the oligodynamic backing sheet segments. Backing sheet materials are made not to absorb or have less absorption over the gauze material. The segmented backing sheet structure can distribute fluid flow throughout the bandage. Which will not make one certain area condensed with fluid from the wound. 098 illustrates the absorbent portion of the gauze pad and 099 illustrates a second absorbent portion can be used above the oligodynamic backing sheet segments. The following embodiments can be manufactured with or without the second gauze pad absorbent portion 099. 101 illustrates how the oligodynamic backing sheet segments and absorbent portions of the gauze pad 098 & 099 can be joined with an adhesive or sewed together. 102 illustrates the spacing the of oligodynamic backing sheet segments. These spacing can be 1 mm apart or larger, 1 mm-3 mm, 2-6 mm, 2-15 mm, 1-15 mm, and 1-18 mm. Not limited to other sizes if the spacing is equal or larger than the porous or mesh structure of some absorbent materials that gauze pads can be made from.

The following embodiments can be made with or without an oligodynamic backing sheet periphery. This oligodynamic backing sheet segment and perforations can be made from none oligodynamic materials such as polymers, fabrics, cotton, polyester, hemp, nylon, bamboo, latex, rubber and other common materials associated with bandage manufacturing of backing sheets with low absorbent properties. Also not limited to having disinfections, antiseptics, antimicrobial, antibacterial and a combination of the following on the backing sheet. Some examples that can be used on the backing sheet and not limited to are hydrocolloid, antibiotic ointment, neomycin, polymyxin, bacitracin, iodine, chlorhexidine, medical honey, antiseptics, witch hazel, alcohol, betadine, saline, and hydrogen peroxide. The oligodynamic backing sheet segments and perforations can be offset with one another and not in a uniform structure.

FIG. 51 Illustrates oligodynamic backing sheet segments are aligned in a uniform structure. 100 illustrates the shape of the oligodynamic backing sheet segments. FIGS. 52 & 53 illustrates a side view of the oligodynamic backing sheet segments with an absorbent gauze pad placed at the bottom, top or both. FIG. 54 illustrates the oligodynamic backing sheet segments fitted into the gauze pad portion 098 and 099. The oligodynamic backing sheet segments can be fitted into one absorbent gauze layer or fitted in between two absorbent gauze layers. An adhesive or sewed layer 103 can be used to hold the oligodynamic backing sheet segments and absorbent gauze layers together. FIG. 54 is not limited to having a periphery oligodynamic backing sheet associated with the above embodiments. Above embodiments are not limited to a backing sheet layer with a combination of perforated or segments on the backing sheet. The above embodiments are not limited to multilayer perforated or segments on the backing sheet.

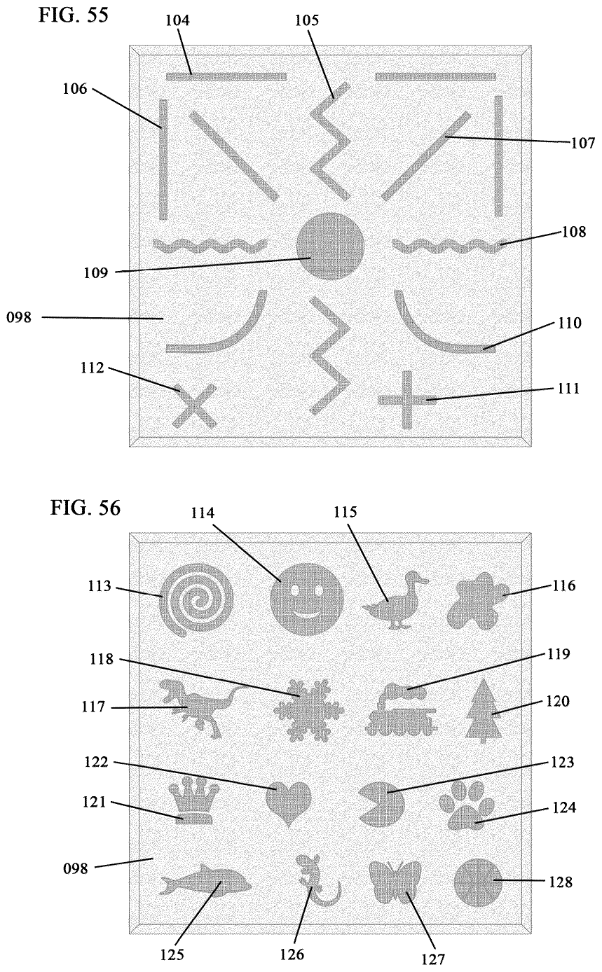

FIG. 55 illustrates various lines and curves that segments of the oligodynamic backing sheet can be made into. 104 illustrates a horizonal line segment, 105 illustrates a zig-zag pattern, 106 illustrates a vertical line segment, 107 illustrates a diagonal line segment, 108 illustrates a wave line, 109 illustrates a circle segment, 110 illustrates a curved or arc pattern, 111 & 112 illustrates a cross pattern. The above embodiments are not limited to various thicknesses, widths, lengths and shapes. The above embodiments are also not limited to various combinations, dashes, segments, patterns and spacing throughout the gauze pad. The above embodiments can be joined together by an adhesive or sewed together onto the gauze pad. The above embodiments can also be made into a perforated form of the shapes illustrated.

FIG. 56. Illustrates various animals, people, emojis, spirals, sea creatures, characters, and objects the oligodynamic backing sheet can be made into. 113 illustrates a spiral design, this design is not limited to various spiral shapes. 114 illustrates a smiley or emoji and is not limited to various impressions and other emoji designs. 115 illustrates a duck and it is not limited to various animals. 116 illustrates an odd shaped object and limited to other formations. 117 illustrates a dinosaur and not limited to various dinosaurs. 118 illustrates a snowflake and not limited to various designs or not limited to holiday designs and objects. 119 illustrates a train and is not limited to boats, automobiles, planes, and any transportation object. 120 illustrates a tree and is not limited to various trees and plants. 121 illustrates a crown, 122 illustrates a heart, and 124 illustrates a paw print. These illustrations are not limited to various objects and characters. 123 illustrates Pac-man character and not limited to other trademark characters or company trademarks. 125 illustrates a dolphin and not limited to other sea creatures. 126 illustrates a lizard and not limited to other reptile creatures. 127 illustrates a butterfly and is not limited to other insect and bug creatures. 128 illustrates basketball and is not limited to other sports and sport team logos. The above embodiments are not limited to various thicknesses, widths, lengths and shapes. The above embodiments are also not limited to various combinations or spacing throughout the gauze pad. The above embodiments can be joined together by an adhesive or sewed together onto the gauze pad. The above embodiments can also be made into a perforated form of the shapes illustrated.

Embodiments of FIGS. 39-56 the backing sheet portion can be made without any oligodynamic materials on it. Since standard backing sheets have little to no absorption properties, they still can be used to distribute fluids throughout the gauze pad without any oligodynamic materials acting as a disinfectant. The segment spacing or perforations can be 1 mm apart or larger, 1 mm-3 mm, 2-6 mm, 2-15 mm, 1-15 mm, and 1-18 mm. Not limited to other sizes if the spacing is equal or larger than the porous or mesh structure of some absorbent materials that gauze pads can be made from.

Following embodiments backing sheet can contain 30%-60%, 10%-35%, less than 50% or larger than 50% oligodynamic coated threads. Following embodiments backing sheet segments can be spaced 2 mm or larger and 3 mm or larger. Following embodiments backing sheet perforations can be 2 mm or larger and 3 mm or larger.

1. A bandage device comprising: a fabric or cloth like backing sheet portion and an absorption pad portion. Where the backing sheet has less absorption properties over the absorption pad portion. The backing sheet comprising of oligodynamic coated threads are greater than 0.02μ (microns) and less than 6μ (microns) of coating thickness on the threads. Where the coated threads width that creates the backing sheet can range from 0.01 mm-1 mm.

2. A bandage comprising a cloth layer backing sheet portion comprising two side tab portions and one central portion provided.
An adhesive layer provided upon the bottom surface of each side tab portions of the cloth layer portion and configured to adhere the bandage to the skin around the wound site with contacting the wound site;
An adhesive layer provided upon the bottom surface of each side tab portions of the cloth layer portion and configured to adhere the bandage to the skin around the wound site with contacting the wound site;
An absorption pad portion provided on a bottom surface of the central portion of the cloth layer portion such that the absorbent pad is operable to contact the wound site of the wearer and separate the cloth layer portion from the wound site of the wearer during use of the bandage.
Where the cloth layer backing sheet portion comprising of oligodynamic coated threads are greater than 0.02μ (microns) and less than 6μ (microns) of coating thickness on the threads. Where the coated threads on the backing sheet have a width of 0.01 mm-1 mm.

3. A bandage gauze comprising a backing sheet layer portion joined with an absorption material layer adhered at the bottom.
Where the backing sheet layer portion comprising of oligodynamic coated threads are greater than 0.02μ (microns) and less than 6μ (microns) of coating thickness on the threads. Where the backing sheet portion has less absorption properties over the absorption layer. Where the backing sheet layer portion is perforated, and these perforations are 1 mm or larger.

4. A bandage gauze comprising a backing sheet layer portion joined together with an absorption material layer adhered at the bottom.
Where the backing sheet layer portion comprising of oligodynamic coated threads are greater than 0.02μ (microns) and less than 6μ (microns) of coating thickness on the threads. Where the backing sheet portion has less absorption properties over the absorption layer. Where the backing sheet layer portion is segmented, and these segments are spaced 1 mm or larger.

5. Where the above claims have an absorbent layer located above and under the oligodynamic perforated or segmented backing sheet layer.

6. Where the above claims can have an oligodynamic backing sheet periphery or a non-oligodynamic backing sheet periphery.

7. Where the oligodynamic backing sheet also comprises non-coated threads with less absorption properties over the absorption pad portion next to the coated oligodynamic threads.

8. Where the oligodynamic backing sheet also comprises of non-coated threads with less absorption properties over the absorption pad portion next to the oligodynamic coated threads. Where 50% or more of the bandage backing sheet threads have an oligodynamic coating on it.

9. Where the oligodynamic backing sheet also comprises of non-coated threads with less absorption properties over the absorption pad portion next to the oligodynamic coated threads. Where 50% or less of the bandage backing sheet threads have an oligodynamic coating on it.

10. Where the oligodynamic backing sheet has a combination of perforated and segments.

11. Where the oligodynamic backing sheet is a multilayer of perforated or segments or a multilayer and combinations perforated and segments

12. Where the above claims backing sheet perforations are larger than the porous or mesh structure of some absorbent materials that gauze pads can be made from. That these perforations are at least 1 mm or larger on the backing sheet and the backing sheet does not have any oligodynamic coating on it.

13. Where the above claims backing sheet segments are spaced larger than the porous or mesh structure of some absorbent materials that gauze pads can be made from. That these segments are spaced at least 1 mm or larger on the backing sheet and the backing sheet does not have any oligodynamic coating on it.

14. Where the above claims where the backing sheet and bandage can be made out of various shapes and sizes

15. Where the above claims can be made into individual bandages or in a roll form

16. Where the above claims are classified as a class 1 or class 2 device under the FDA. Where the device has a product usage of 48 hours or less.

17. Where the oligodynamic coated threads can be a combination of various oligodynamic threads and not limited to one particular coating style.

18. Oligodynamic backing sheet is not limited to fabric bandages and can be made into polymers type bandages.

19. Where the backing sheet perforations are 2 mm and larger or the backing sheet segments are spaced 2 mm or larger.

20. Where the backing sheet perforations are 3 mm and larger or the backing sheet segments are spaced 3 mm or larger.
